(19) United States
(12) Patent Application Publication
(10) Pub. No.: US 2016/0375035 A1
(43) Pub. Date: Dec. 29, 2016

(54) CHROMIUM HISTIDINATE AND CHROMIUM PICOLINATE COMPLEXES

(71) Applicant: JDS Therapeutics, LLC, Purchase, NY (US)

(72) Inventor: James R Komorowski, Trumbull, CT (US)

(21) Appl. No.: 15/188,231

(22) Filed: Jun. 21, 2016

Related U.S. Application Data
(60) Provisional application No. 62/183,645, filed on Jun. 23, 2015.
(60) Provisional application No. 62/183,611, filed on Jun. 23, 2015.

(51) Int. Cl. A61K 31/555; A23L 33/10

(52) U.S. Cl. CPC A61K 31/555; A23L 33/10; A23V 2002/00

(57) ABSTRACT

This present disclosure generally relates to the discovery that chromium histidinate (CrHis) and chromium picolinate (CrPic) can exist as complexes in multiple forms. More particularly, the present disclosure is directed to the surprising discovery that particular forms/isomers (or combinations of forms/isomers) of such chromium complexes have greater activity than others.

### CROSS-REFERENCE TO RELATED APPLICATIONS

This application claims priority to U.S. Provisional Patent Application No. 62/183,611, entitled “CHROMIUM PICOLINATE COMPLEXES,” filed Jun. 23, 2015, and U.S. Provisional Patent Application No. 62/183,645, entitled “CHROMIUM HISTIDINATE COMPLEXES,” filed Jun. 23, 2015, the entire contents of which are hereby incorporated by reference in their entirety.

### BACKGROUND

### Field

This present disclosure generally relates to the discovery that chromium histidinate (CrHis) and chromium picolinate (CrPic) can exist as complexes in multiple forms. More particularly, the present disclosure is directed to the surprising discovery that particular forms/isomers (or combinations of forms/isomers) of such chromium complexes have greater activity than others.

Chromium is a nutritionally essential trace element. Chromium is essential for optimal insulin activity in all known insulin-dependent systems (Boyle, et al, Southern Med. J. (1977) 70:1449-1453). Insufficient dietary chromium has been linked to both maturity-onset diabetes and to cardiovascular disease.

Chromium functions as a cofactor for insulin. It binds to the insulin receptor and potentiates many, and perhaps all, of its functions. These functions include, but are not limited to, the regulation of carbohydrate and lipid metabolism.

Many diseases and disorders have been associated—etiologically or otherwise—to impaired, altered, or abnormal glucose metabolism. These diseases and disorders include, but are not limited to: diabetes (hyperglycemia); hypoglycemia; cardiometabolic syndrome; Alzheimer's disease; Huntington's disease; epilepsy; ischemia; Parkinson's disease; amnesia; dementia; mild cognitive impairment (MCI); attention deficit hyperactivity disorder (ADHD); amyotrophic lateral sclerosis (ALS); and, traumatic brain injury.

Hypoglycemia is a term that literally means “low blood sugar.” Hypoglycemia includes a state of a blood glucose level of not higher than about 60 mg/dL, but is not limited to this blood glucose level. For example, when a person having high blood glucose due to diabetes or the like undergoes a reduction in blood glucose level upon insulin injection or the administration of an antidiabetic agent, or when a healthy individual undergoes rapid reduction in blood glucose level due to hunger or strenuous exercise, similar conditions to hypoglycemia can appear even at about 100 mg/dL. Hypoglycemia often arises as a side effect of diabetes treatment (e.g., administration of insulin). Hypoglycemia can also result, however, from other medications or diseases, hormone or enzyme deficiencies, or tumors. Furthermore, hypoglycemia can result from a long-term habit of ingesting large amounts of carbohydrates; from excessive ingestion of alcohol; and from continuation of extreme exercise for a long time in a state of dietary insufficiency. Hypoglycemia induced by diabetes treatment or other medications are particularly dangerous, however, resulting in a higher probability of a severe condition as compared to other causes of hypoglycemia.

Hypoglycemia-related disorders and hypoglycemia-related complications refer to conditions or complications that arise as a result of low blood sugar, such as insulin-induced brain tissue damage, and the like. Hypoglycemia-related disorders and hypoglycemia-related conditions may occur where a reduction in glucose level in blood is accompanied by a reduction in glucose level in the brain thereby causing lassitude, general discomfort, dismay, malaise, jitteriness, trembling, headache, weakness, cold sweat and palpitation, additionally causing impaired consciousness and coma, which may also lead to death in a serious case.

Diabetes mellitus is known to affect at least 10 million Americans, and millions more may unknowingly have the disease. Diabetes is the sixth leading cause of death in the United States and accounted for more than 193,000 deaths in 1997. Diabetes is a disease state in which the pancreas does not release insulin at levels capable of controlling glucose levels. Diabetes is classified into two types. The first type is diabetes (Type 1) that is insulin dependent and usually appears in young people. The islet cells of the pancreas stop producing insulin mainly due to autoimmune destruction. Standard therapy for Type 1 diabetes is the administration of insulin. Type 1 diabetic patients are the minority of total diabetic patients (up to 10% of the entire diabetic population). The second type of diabetes (Type 2) is non-insulin dependent diabetes, which is caused by a combination of insulin resistance and insufficient insulin secretion. This is the most common type of diabetes in the Western world. Close to 8% of the adult population of various countries around the world, including the United States, have Type 2 diabetes, and about 30% of these patients will need to use insulin at some point during their life span due to secondary pancreas exhaustion.

The American Diabetes Association (ADA), World Health Organization (WHO) and Japan Diabetes Society (JDS) recently announced new diagnostic criteria for diabetes, taking into consideration the achievements of clinical and epidemiologic studies. Under these criteria, one is classified as diabetic when any of the following blood glucose levels are observed: fasting blood glucose ≧126 mg/dL; casual blood glucose ≧200 mg/dL; or blood glucose two hours after the 75 g oral glucose tolerance test (OGTT) ≧200 mg/dL (Diabetes Care 20: 1183 (1997); Diabet Med 15: 539 (1998); and Diabetes 42: 385 (1999)).

Post-prandial hyperglycemia is another blood glucose related disorder that can occur in both diabetic and non-diabetic individuals. Post-prandial hyperglycemia is characterized by higher spikes in blood glucose levels after consuming food and/or beverages in comparison to a normal individual. Certain individuals may suffer from post-prandial hyperglycemia, but not exhibit symptoms for months, even years, while the high blood glucose levels are damaging certain tissues and organ systems, such as the kidneys. Thus, there is a need for improved methods to blunt these abnormally high spikes in blood glucose levels seen in individuals suffering from post-prandial hyperglycemia.

Glucose homeostasis is critical for energy generation, neuronal maintenance, neurogenesis, neurotransmitter regulation, cell survival and synaptic plasticity. Glucose is the principle energy source for mammalian brain, and a key role in cognitive function.

Delivery of glucose from the blood to the brain requires its transport across the endothelial cells of the blood-brain barrier and across the plasma membranes of neurons and glia, which is mediated by the facilitative glucose transporter proteins. Facilitative glucose transport is mediated by one or more members of the closely-related glucose transporter (GLUT) family. Thirteen members of the GLUT family have been described thus far. Tissue-specific glucose transporters allocate glucose among organs in order to maintain brain glucose concentrations. The two primary glucose transporter isoforms which function in cerebral glucose metabolism are GLUT-1 and GLUT-3. GLUT-1 is the primary transporter in the blood-brain barrier, choroid plexus, ependyma, and glia; GLUT-3 is the neuronal glucose transporter. GLUT-4, on the other hand, carries glucose across the membranes of muscle and fat cells.

Insulin, a regulator of glucose uptake, is secreted by the pancreas. Insulin allocates glucose to muscle and fat. The hypothalamus-pituitary-adrenal (HPA) axis, the sympathetic nervous system (SNS), and vascular endothelial growth factor allocate glucose to the brain. Feedback pathways both from the brain and from muscle and fat are involved in regulating glucose allocation and exogenous glucose supply. Further, insulin can cross the blood-brain barrier (BBB), reaching neurons and glial cells, and can exert a region-specific effect on glucose metabolism. Increased glucose consumption causes an increase in the net transport of glucose from blood to brain. It has been shown that insulin-induced hypoglycemia increases brain GLUT-1 & GLUT-3 levels. (Uehara et al., (1997) Am. J. Physiol. 272:E716-E719). Thus, insulin indirectly affects the transport without acting on the transport mechanisms. It has been proposed that part of the insulin action may take place in extracerebral tissues via changes of the amino acid balance in the blood. (Reagan et al., (1999) Am. J. Physiol. Endocrinol. Metab. 276:E879-E886).

GLUT-1 facilitates transport of glucose across the blood-brain-barrier. GLUT-1 expression levels are insulin-independent. Rather, GLUT-1 is dependent on potent regulators of blood vessel function like vascular endothelial growth factor (VEGF), a pituitary counter regulatory hormone. HPA-axis overdrive causes metabolic abnormalities such as central adiposity, hyperglycemia, dyslipidemia, and hypertension, that are well known clinical aspects the metabolic syndrome. Overexpression of GLUT-1 in skeletal muscle is associated with marked increases in lactate and glycogen due to an increase in basal glucose uptake, and increased glucose flux results in resistance of GLUT-4 to activation by insulin and other stimuli, such as hypoxia and contractile activity (Katsumata et al., (1999) FASEB J. 11:1405-13).

GLUT-3, the neuron-specific glucose transporter, is solely responsible for the delivery of glucose into neurons in the central nervous system. GLUT-3 mRNA is widely expressed in the brain, including the pyramidal neurons of the hippocampus, the granule neurons of the dentate gyrus, and the cortex.

Brain-specific kinases 1 and 2 (BRSK1/2) are AMP-activated protein kinase (AMPK)-related kinases that are highly expressed in mammalian forebrain. The activation of AMPK plays an important, albeit not an exclusive, role in the induction of recruitment of the insulin-dependent glucose transporter found in skeletal muscle, GLUT-4, to the plasma membrane. The ability of AMPK to stimulate GLUT-4 translocation to the plasma membrane in skeletal muscle occurs via a mechanism distinct from that stimulated by insulin since together insulin and AMPK effects are additive. In addition to its role in the regulation of GLUT-4, data suggest that AMPK regulates glucose transport through GLUT-1.

Altered glucose metabolism in the brain is associated with various disease states, including but not limited to Alzheimer's disease, Huntington's disease, epilepsy, ischemia, amnesia, and traumatic brain injury. Glucose transporter expression is believed to be related to altered glucose metabolism. Chronic hyperglycemia downregulates GLUT-1 and GLUT-3 expression at both mRNA and protein levels in the brain, which is not due to the decrease of the density of microvessels. (Hou et al., (2007) Chin. Med. J (Engl). 120(19):1704-1709). The downregulation of GLUT-1 and GLUT-3 expression might be the adaptive reaction of the body to prevent excessive glucose entering the cell that may lead to cell damage. Studies suggest that chronic stress produces molecular, morphological, and ultrastructural changes in the hippocampus that are accompanied by cognitive deficits. Further, in insulin resistance, dementia, and cognitive impairment, and Alzheimer's disease, there is a reduced sensitivity to insulin resulting in hyperinsulinemia. Toxic levels of insulin negatively influence neuronal function and survival, and elevation of peripheral insulin concentration acutely increases its cerebrospinal fluid (CSF) concentration. Peripheral hyperinsulinemia correlates with an abnormal removal of the amyloid beta peptide (Abeta) and an increase of tau hyperphosphorylation as a result of augmented cdk5 and GSK3beta activities. This leads to cellular cascades that trigger a neurodegenerative phenotype and decline in cognitive function.

GLUT-1 deficiency syndrome is a disorder that primarily affects the brain. Affected individuals generally have seizures beginning in the first few months of life. Infants with GLUT-1 deficiency syndrome have a normal head size at birth, but growth of the brain and skull is often slow, in severe cases resulting in an abnormally small head size (microcephaly). Subjects with GLUT-1 deficiency syndrome often exhibit developmental delay or intellectual disability. GLUT-1 deficiency syndrome is also associated with other neurological problems, such as stiffness caused by abnormal tensing of the muscles (spasticity), difficulty in coordinating movements (ataxia), and speech difficulties (dysarthria). Some experience episodes of confusion, lack of energy (lethargy), headaches, muscle twitches (myoclonus), or involuntary irregular eye movements, particularly before meals.

Dietary supplementation of chromium to normal individuals has been reported to lead to improvements in glucose tolerance, serum lipid concentrations, including high-density lipoprotein cholesterol, insulin and insulin binding (Cefalu et al., (2004) Diabetes Care 27(10:2741-51). Supplemental chromium in the trivalent form, e.g. chromic chloride, is associated with improvements of risk factors associated with adult-onset (Type 2) diabetes and cardiovascular disease.

The introduction of inorganic chromium compounds per se into individuals is not particularly beneficial. Chromium must be converted endogenously into an organic complex or must be consumed as a biologically active molecule. Only about 0.5% of ingested inorganic chromium, however, is assimilated into the body. Recommended Daily Allowances, Ninth Revised Edition, Nat. Acad. Sci., page 160, 1980. Only 1-2% of most organic chromium compounds are assimilated into the body.

U.S. Pat. No. Re. 33,988 discloses that when selected essential metals, including chromium, are administered to mammals as exogenously synthesized coordination complexes of picolinic acid, they are directly available for absorption without competition from other metals. This patent describes a composition and method for selectively supplementing the essential metals in the human diet and for facilitating absorption of these metals by intestinal cells. These complexes are safe, inexpensive, biocompatible, and easy to produce. These exogenously synthesized essential metal coordination complexes of picolinic acid (pyridine-2-carboxylic acid) have the following structural formula:

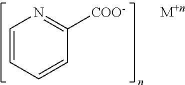

wherein M represents the metallic cation and n is equal to the cation's valence. For example, when M is Cr and n=3, then the compound is chromic tripicolinate. Other chromium picolinate and or chromium histidinate and or chromium complex alone and or in combinations disclosed include chromic monopicolinate and chromic dipicolinate.

Picolinic acid form coordination complexes with monovalent, divalent and trivalent metal ions and facilitate the absorption of these metals by transporting them across intestinal cells and into the bloodstream. Chromium absorption in rats following oral administration of CrCl₃ was facilitated by the non-steroidal anti-inflammatory drugs (NSAIDs) aspirin and indomethacin (Davis et al., (1995) J. Nutrition Res. 15:202-210) (Kamath et al., J Nutrition (1997) 127:478-482). These drugs inhibit the enzyme cyclooxygenase which converts arachidonic acid to various prostaglandins, resulting in inhibition of intestinal mucus formation and lowering of intestinal pH which facilitates chromium absorption.

Delivering sustained, safe, and efficacious amounts of micronutrients such as chromium is a continuing technical problem. For example, administration of chromium picolinate provides a relatively fast increase in blood chromium levels. However, the blood chromium levels peak quickly and then fall back to normal levels. In contrast, chromium histidinate is absorbed much more slowly from the gastrointestinal tract, providing a detectable increase in blood chromium levels only hours after administration. Accordingly, there is a need for better forms of chromium to provide sustained therapeutic blood chromium levels.

There is a need for improved therapies for disorders and diseases associated with altered glucose levels and/or metabolism. Chromium and chromium complexes may play a significant role in the development and application of such improved therapies. However, there remains a need for better forms of chromium to enhance delivery and bioavailability, and have a longer lasting effect.

### SUMMARY

Some embodiments provide a formulation comprising at least about 5% (w/w) chromium histidinate isomer 1, having the structure:

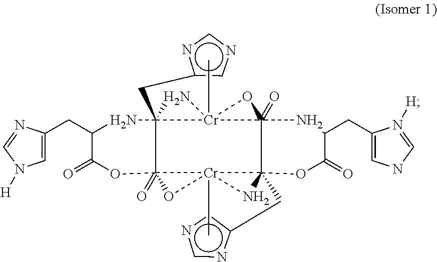

at least about 15% (w/w) chromium histidinate isomer 2, having the structure:

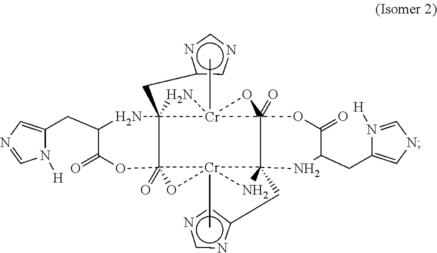

and at least about 45% (w/w) chromium histidinate isomer 3, having the structure:

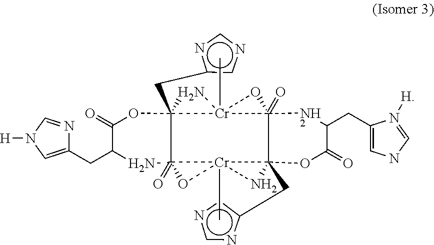

In some embodiments, the formulation contains no more than about 30% (w/w) free histidine. In some embodiments, the formulation contains no more than about 20% (w/w) free histidine. In some embodiments, the formulation contains no more than about 10% (w/w) free histidine. In some embodiments, the formulation contains no more than about 8% (w/w) free histidine.

In some embodiments, the formulation comprises about 8% of chromium histidinate isomer 1, about 20% of chromium histidinate isomer 2, about 50% of chromium histidinate isomer 3, and not more than about 8% free histidine. In some embodiments, the formulation further comprises chromium picolinate. In some embodiments, the formulation further comprises at least one nutritionally acceptable carrier, excipient, or diluent.

Some embodiments provide a formulation comprising no more than 10% (w/w) chromium histidinate isomer 1, having the structure:

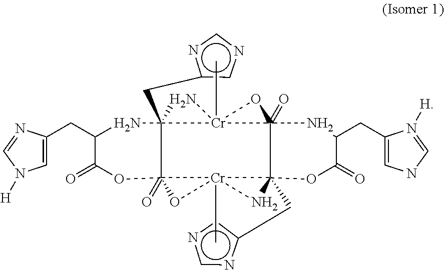

In some embodiments, the formulation contains no more than 10% (w/w) free histidine. In some embodiments, the formulation contains no more than 8% (w/w) free histidine. In some embodiments, the formulation further comprises at least 15% (w/w) chromium histidinate isomer 2, having the structure:

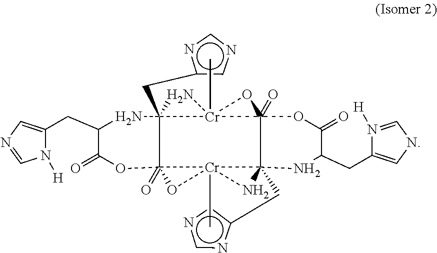

In some embodiments, the formulation further comprises at least 30% (w/w) chromium histidinate isomer 3, having the structure:

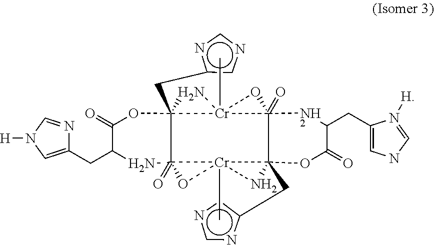

In some embodiments, the formulation further comprises at least one nutritionally acceptable carrier, excipient, or diluent. In some embodiments, the formulation includes at least 40% (w/w) chromium histidinate isomer 3.

### BRIEF DESCRIPTION OF THE DRAWINGS

FIG. 1A is a UV chromatogram trace of chromium histidinate (“CrHis”) solution run through an HPLC. The CrHis solution used in FIG. 1A was about one week old.

FIG. 1B is a TIC (total ion current) chromatogram trace of the same CrHis solution as used in FIG. 1A.

FIG. 2A is a UV chromatogram trace of CrHis solution through HPLC. The CrHis solution used in FIG. 2A was prepared fresh.

FIG. 2B is a TIC chromatogram trace of the same CrHis solution as used in FIG. 2A.

FIG. 3A is a mass spectrum plot of the peak seen at about 29.5 minutes in FIGS. 1A-1B (“Peak 1”).

FIG. 3B is a mass spectrum plot of the peak seen at about 32.5 minutes in FIGS. 1A-1B (“Peak 2”).

FIG. 3C is a mass spectrum plot of the peak seen at about 39.5 minutes in FIGS. 1A-1B (“Peak 3”).

FIG. 4A depicts the structure of a CrHis isomer according to one embodiment (hereinafter “Isomer 1”).

FIG. 4B depicts the structure of a CrHis isomer according to one embodiment (hereinafter “Isomer 2”).

FIG. 4C depicts the structure of a CrHis isomer according to one embodiment (hereinafter “Isomer 3”).

FIG. 5A is a UV chromatogram trace of chromium picolinate (“CrPic”) solution run through an HPLC.

FIG. 5B is a TIC (total ion current) chromatogram trace of the same CrPic solution as used in FIG. 5A.

FIG. 6 is a mass spectrum plot of the peak seen at about 10.1 minutes in FIGS. 5A-5B.

FIG. 7A depicts the structure of a CrPic isomer according to one embodiment (hereinafter “CrPic Isomer 1”).

FIG. 7B depicts the structure of a CrPic isomer according to one embodiment (hereinafter “CrPic Isomer 2”).

FIG. 8 is a bar graph depicting insulin levels after administration of various formulations containing various amounts of CrHis isomers.

FIG. 9A is a bar graph depicting GLUT-6 levels in ovaries from rats treated with various Cr-His formulations. CrHis4 refers to a mixture of CrHis isomers 1, 2, and 3.

FIG. 9B is a bar graph depicting GLUT-4 levels in ovaries from rats treated with various Cr-His formulations. CrHis4 refers to a mixture of CrHis isomers 1, 2, and 3.

### DETAILED DESCRIPTION

Chromium complexes such as, for example, chromium histidinate (CrHis) and chromium picolinate (CrPic) can be components of vitamins and nutraceuticals. However, there is a need for chromium forms, formulations and/or compositions that have improved activity, increased efficacy, and/or longer lasting effects.

The present disclosure relates to the discovery that at least chromium histidinate complexes and chromium picolinate complexes can exist in multiple forms, conformations, isomers, and/or crystal structures. Moreover, the present disclosure relates to the surprising discovery that certain of these forms have unexpectedly advantageous properties over the other forms and/or over the prior CrHis compositions and/or formulations.

Compositions and/or formulations having various amounts of one or more forms of CrHis/CrPic may be made and administered. Compositions having a higher amount of a particular CrHis/CrPic form can have more activity than compositions in the prior art. For example, one or more forms may be purified, concentrated, and or adjusted. Thus, a composition having, for example, >90% (w/w) of a particular form of CrHis may be obtained. Such purified compositions and/or formulations may have greater activity than prior art compositions and/or formulations. For example, a formulation may include 99% (w/w) or greater than 99% (w/w) of one particular form and have no other forms included in the formulation (e.g., about 99% (w/w) of one particular form and about 0% (w/w) of any other form). In other example, a formulation having about 50% (w/w) of a first form and about 50% (w/w) of a second form and no other form may be made and administered. Compositions with different ratios of isomers can also have more activity than compositions in the prior art. For example, one or more isomers may be enriched, or depleted. For example, a formulation may include less than 15% of CrHis isomer 1, more than 12% CrHis isomer 2, and more than 22% CrHis isomer 3.

Varying the concentrations and/or ratios of the various forms of CrHis/CrPic can lead to compositions and/or formulations that have greater activity than prior art compositions and/or formulations. For example, a composition and/or formulation having about 50% (w/w) of one form of CrHis, about 20% (w/w) of a second form of CrHis, and about 10% (w/w) of a second form of CrHis may have greater activity than prior art compositions and/or formulations.

In some embodiments, the formulation is substantially free of free histidine, or a salt thereof. In some embodiments, the formulation includes at most about 30% (w/w) free histidine, or a salt thereof. In some embodiments, the formulation includes at most about 25% (w/w) free histidine, or a salt thereof. In some embodiments, the formulation includes at most about 20% (w/w) free histidine, or a salt thereof. In some embodiments, the formulation includes at most about 15% (w/w) free histidine, or a salt thereof. In some embodiments, the formulation includes at most about 12% (w/w) free histidine, or a salt thereof. In some embodiments, the formulation includes at most about 10% (w/w) free histidine, or a salt thereof. In some embodiments, the formulation includes at most about 8% (w/w) free histidine, or a salt thereof. In some embodiments, the formulation includes at most about 5% (w/w) free histidine, or a salt thereof. In some embodiments, the formulation includes at most about 4% (w/w) free histidine, or a salt thereof. In some embodiments, the formulation includes at most about 3% (w/w) free histidine, or a salt thereof. In some embodiments, the formulation includes at most about 2% (w/w) free histidine, or a salt thereof. In some embodiments, the formulation includes at most about 1% (w/w) free histidine, or a salt thereof.

The present disclosure also reveals that different methods of manufacturing CrHis/CrPic can lead to more or less of the various forms of CrHis/CrPic being present in a resulting sample. Such methods of manufacture can result in formulations with more of an active form of CrHis than prior art methods of manufacture.

The embodiments disclosed herein are based, in part, upon the surprising discovery of novel forms of chromium histidinate complexes that have improved therapeutic efficacy and benefits. Thus, in accordance with the embodiments described herein, provided are compositions for the improved delivery of chromium, and uses thereof.

The embodiments disclosed herein are based, in part, upon the surprising discovery of novel forms of chromium picolinate complexes that have improved therapeutic efficacy and benefits. Thus, in accordance with the embodiments described herein, provided are compositions for the improved delivery of chromium, and uses thereof.

Some embodiments provide novel crystalline forms of chromium histidinate. Some embodiments provide novel isomers of chromium histidinate. Some embodiments provide novel stable conformers of chromium histidinate. Some embodiments provide combinations of the forgoing.

Some embodiments provide novel crystalline forms of chromium picolinate. Some embodiments provide novel isomers of chromium picolinate. Some embodiments provide novel stable conformers of chromium picolinate. Some embodiments provide combinations of the forgoing.

Some embodiments provide formulations comprising mixtures of novel crystalline forms of CrHis and CrPic. Some embodiments provide formulations comprising mixtures of novel isomers of CrHis and CrPic. Some embodiments provide novel stable conformers of chromium picolinate.

In various cases, a ligand(s) has/have the ability to bond to chromium via its carboxylate functional group as well as through pi electron-d orbital interaction. This secondary interaction between the ligand and chromium can increase the bioavailability and absorption of chromium.

While the chromium complexes aid in the absorption of chromium by intestinal cells, in some embodiments, uncomplexed chelating agents are advantageously included in the compositions to facilitate absorption of other ingested chromium as well as other metals including, but not limited to, copper, iron, magnesium, manganese, and zinc. Suitable chelating agents include histidine, tri-histidine, picolinic acid.

Certain forms of CrHis complexes may be absorbed by various tissues and/or cells faster and/or to a greater extent than other forms. For example, certain forms of CrHis complexes may be absorbed faster and/or to a greater extent in intestinal cells than others. Certain forms of CrHis complexes may have greater activity after being absorbed than other forms. For example, certain forms of CrHis complexes may cause cells to uptake glucose faster and/or to a greater extent than other forms. In other implementations, certain forms of CrHis complexes may cause Cr concentrations in the blood, cells, and/or tissues to rise faster and/or to a greater extent than other forms. In other implementations, certain forms of CrHis complexes may cause glucose transporters to increase activity and/or increase in concentration faster and/or to a greater extent than other forms.

Likewise, certain forms of CrPic complexes may be absorbed by various tissues and/or cells faster and/or to a greater extent than other forms. For example, certain forms of CrPic complexes may be absorbed faster and/or to a greater extent in intestinal cells than others. Certain forms of CrPic complexes may have greater activity after being absorbed than other forms. For example, certain forms of CrPic complexes may cause cells to uptake glucose faster and/or to a greater extent than other forms. In other implementations, certain forms of CrPic complexes may cause Cr concentrations in the blood, cells, and/or tissues to rise faster and/or to a greater extent than other forms. In other implementations, certain forms of CrPic complexes may cause glucose transporters to increase activity and/or increase in concentration faster and/or to a greater extent than other forms.

Chelating agents such as histidine and picolinic acid are available from many commercial sources, including Sigma-Aldrich (St. Louis, Mo.) (picolinic acid; catalog No. P5503). In some embodiments, the ratio of the chromium complex to the chelating agent in the embodiments disclosed herein can be from about 10:1 to about 1:10 (w/w), more preferably from about 5:1 to about 1:5 (w/w), e.g., 5:1, 5:2, 5:3, 5:4, 1:1; 1:2, 1:3, 1:4, 1:5, or any number in between. Alternatively, the molar ratio of chromium complex to the uncomplexed chelating agent is preferably 1:1, and can be from about 5:1 to about 1:10, e.g., e.g., 5:1, 5:2, 5:3, 5:4, 1:1; 1:2, 1:3, 1:4, 1:5, 1:6, 1:7, 1:8, 1:9, 1:10, or any number in between. In some embodiments, the chelating agent is one or more D- or L-amino acids. In some embodiments, the one or more amino acids may form a mono-, di-, or tri-chromium complex. In some embodiments, the one or more amino acids may form a mono-, di-, or tri-amino acid complex with the chromium. In some embodiments, the one or more amino acids include, but are not limited to, chromium histidine, chromium di-hisitidine chromium tri-histidine, chromium poly-hisitidine, chromium picolinate, chromium di-picolinate, chromium tri-picolinate, and chromium polypicolinate.

Some embodiments provide compositions and methods of treating subjects with compositions that comprise, consist essentially of, and/or consist of a therapeutically effective amount of chromium. Some embodiments provide compositions and methods of treating subjects with compositions that comprise, consist essentially of, or consist of CrHis or CrPic in combination with one or more additional agents. Various methods of treatment are discussed below. Some embodiments provide compositions and methods of treating subjects with compositions that comprise, consist essentially of, or consist of CrHis or CrPic having various amounts, ratios, and/or combinations, of one or more of the particular form of the CrHis or CrPic complex.

A “therapeutically effective amount” as used herein includes within its meaning a non-toxic but sufficient amount of a compound active ingredient or composition comprising the same for use in the embodiments disclosed herein to provide the desired therapeutic effect. The exact amount of the active ingredient disclosed herein required will vary from subject to subject depending on factors such as the species being treated, the age and general condition of the subject, the severity of the condition being treated, the particular agent being administered, the weight of the subject, and the mode of administration and so forth. Thus, it is not possible to specify an exact “effective amount”. However, for any given case, an appropriate “effective amount” may be determined by one of ordinary skill in the art using only routine methods.

By way of example, a “therapeutically effective amount” of the chromium disclosed herein can be, for example, 0.001 μg/kg, 0.01 μg/kg, 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 80 μg/kg 0, 850 μg/kg, 900 μg/kg, 1 mg/kg, 1.5 mg·kg, 2.0 mg/kg, 2.5 mg/kg, 3 mg/kg, 4.0 mg/kg, 5.0 mg/kg, 6 mg/kg, 7 mg/kg, 8 mg/kg, 9 mg/kg, 10 mg/kg, 15 mg/kg, 20 mg/kg, 25 mg/kg, 30 mg/kg, 35 mg/kg, 40 mg/kg, 45 mg/kg 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 125 mg/kg, 150 mg/kg, 200 mg/kg, 250 mg/kg, 300 mg/kg, 350 mg/kg, 400 mg/kg, 450 mg/kg, 500 mg/kg, 550 mg/kg, 600 mg/kg, 650 mg/kg, 700 mg/kg, 750 mg/kg, 800 mg/kg, 850 mg/kg, 900 mg/kg, 950 mg/kg, 1 g/kg, 5 g/kg, 10 g/kg, or more, or any fraction in between of chromium. Accordingly, in some embodiments, the dose of chromium in compositions disclosed herein can be about 0.001 μg to about 100 g, preferably per day. For example, the amount of chromium can be 0.001 μg, 0.01 μg, 0.1 μg, 0.2 μg, 0.3 μg, 0.4 μg, 0.5 μg, 0.6 μg, 0.7 μg, 0.8 μg, 0.9 μg, 1 μg, 2 μg, 3 μg, 4 μg, 5 μg, 6 μg, 7 μg, 8 μg, 9 μg, 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 125 μg, 150 μg, 175 μg, 200 μg, 225 μg, 250 μg, 275 μg, 300 μg, 325 μg, 350 μg, 375 μg, 400 μg, 425 μg, 450 μg, 475 μg, 500 μg, 525 μg, 575 μg, 600 μg, 625 μg, 650 μg, 675 μg, 700 μg, 725 μg, 750 μg, 775 μg, 800 μg, 825 μg, 850 μg, 875 μg, 900 μg, 925 μg, 950 μg, 975 μg, 1000 μg, 1.25 g, 1.5 g, 1.75 g, 2.0 g, 2.25 g, 2.5 g, 2.75 g, 3.0 g, 3.25 g, 3.5 g, 3.5 g, 3.75 g, 4.0 g, 4.25 g, 4.5 g, 4.75 g, 5.0 g, 5.25 g, 5.5 g, 5.75 g, 6.0 g, 6.25 g, 6.5 g, 6.75 g, 7.0 g, 7.25 g, 7.5 g, 7.75 g, 8.0 g, 8.25 g, 8.5 g, 8.75 g, 9.0 g, 8.25 g, 9.5 g, 9.75 g, 10 g, 20 g, 30 g, 40 g, 50 g, 60 g, 70 g, 80 g, 90 g, 100 g, or more, or any range or amount in between any two of the preceding values. The exemplary therapeutically effective amounts listed above, can, in some embodiments be administered in the methods described elsewhere herein on an hourly basis, e.g., every one, two, three, four, five, six, seven, eight, nine, ten, eleven, twelve, thirteen, fourteen, fifteen, sixteen, seventeen, eighteen, nineteen, twenty, twenty-one, twenty-two, twenty-three hours, or any interval in between, or on a daily basis, every two days, every three days, every four days, every five days, every six days, every week, every eight days, every nine days, every ten days, every two weeks, every month, or more or less frequently, as needed to achieve the desired therapeutic effect.

The administration of the one or more of the compositions disclosed herein can be by any of the methods of administration described herein or by delivery methods known by one of skill in the art. The compositions may be administered orally, through parenteral nutrition, e.g., feeding tube or intravenously, and through other known means.

For oral administration, the compositions disclosed herein can be provided as a tablet, aqueous or oil suspension, dispersible powder or granule, emulsion, hard or soft capsule, syrup, elixir, or beverage. Compositions intended for oral use can be prepared according to any method known in the art for the manufacture of pharmaceutically acceptable compositions and such compositions may contain one or more of the following agents: sweeteners, flavoring agents, coloring agents and preservatives. The sweetening and flavoring agents will increase the palatability of the preparation. Tablets containing chromium complexes in admixture with non-toxic pharmaceutically acceptable excipients suitable for tablet manufacture are acceptable. Pharmaceutically acceptable vehicles such as excipients are compatible with the other ingredients of the formulation (as well as non-injurious to the patient). Such excipients include inert diluents such as calcium carbonate, sodium carbonate, lactose, calcium phosphate or sodium phosphate; granulating and disintegrating agents, such as corn starch or alginic acid; binding agents such as starch, gelatin or acacia; and lubricating agents such as magnesium stearate, stearic acid or talc. Tablets can be uncoated or can be coated by known techniques to delay disintegration and absorption in the gastrointestinal tract and thereby provide a sustained action over a longer period of time. For example, a time delay material such as glyceryl monostearate or glyceryl distearate alone or with a wax can be employed.

Formulations for oral use can also be presented as hard gelatin capsules wherein the active ingredient is mixed with an inert solid diluent, for example calcium carbonate, calcium phosphate or kaolin, or as soft gelatin capsules wherein the active ingredient is mixed with water or an oil medium, such as peanut oil, liquid paraffin or olive oil. Aqueous suspensions can contain the chromium complex of the invention in admixture with excipients suitable for the manufacture of aqueous suspensions. Such excipients include suspending agents, dispersing or wetting agents, one or more preservatives, one or more coloring agents, one or more flavoring agents and one or more sweetening agents such as sucrose or saccharin.

Oil suspensions can be formulated by suspending the active ingredient in a vegetable oil, such as arachis oil, olive oil, sesame oil or coconut oil, or in a mineral oil such as liquid paraffin. The oil suspension can contain a thickening agent, such as beeswax, hard paraffin or cetyl alcohol. Sweetening agents, such as those set forth above, and flavoring agents can be added to provide a palatable oral preparation. These compositions can be preserved by an added antioxidant such as ascorbic acid. Dispersible powders and granules of the invention suitable for preparation of an aqueous suspension by the addition of water provide the active ingredient in admixture with a dispersing or wetting agent, a suspending agent, and one or more preservatives. Additional excipients, for example sweetening, flavoring and coloring agents, can also be present.

Syrups and elixirs can be formulated with sweetening agents, such as glycerol, sorbitol or sucrose. Such formulations can also contain a demulcent, a preservative, a flavoring or a coloring agent.

The chromium preparations for parenteral administration can be in the form of a sterile injectable preparation, such as a sterile injectable aqueous or oleaginous suspension. This suspension can be formulated according to methods well known in the art using suitable dispersing or wetting agents and suspending agents. The sterile injectable preparation can also be a sterile injectable solution or suspension in a non-toxic parenterally-acceptable diluent or solvent, such as a solution in 1,3-butanediol. Suitable diluents include, for example, water, Ringer's solution and isotonic sodium chloride solution. In addition, sterile fixed oils can be employed conventionally as a solvent or suspending medium. For this purpose, any bland fixed oil can be employed including synthetic mono or diglycerides. In addition, fatty acids such as oleic acid can likewise be used in the preparation of injectable preparations.

The compositions can also be in the form of oil-in-water emulsions. The oily phase can be a vegetable oil, such as olive oil or arachis oil, a mineral oil such as liquid paraffin, or a mixture thereof. Suitable emulsifying agents include naturally-occurring gums such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsions can also contain sweetening and flavoring agents.

It will be appreciated that the amount of chromium or chromium complex may be combined with a carrier material to produce a single dosage form. Such forms will vary depending upon the host treated and the particular mode of administration.

When administered to a mammal, e.g., to an animal for veterinary use or for improvement of livestock, or to a human for therapeutic use, the compositions disclosed herein are administered in isolated form or as the isolated form in a therapeutic composition. As used herein, “isolated” means that the compositions disclosed herein are separated from other components of either (a) a natural source, such as a plant or cell or food, preferably bacterial culture, or (b) a synthetic organic chemical reaction mixture. Preferably, via conventional techniques, the compositions disclosed herein are purified. As used herein, “purified” means that when isolated, the isolate contains at least 95%, preferably at least 98% of the chromium histidinate and/or chromium picolinate in the composition, not including any free histidine and/or picolinic acid in the composition.

As used herein, “free histidine” refers to histidine, or a salt thereof, that is not complexed with a chromium ion or ions.

As used herein, “identifying,” refers to detecting or selecting a subject from a population of potential subjects, for example, to establish that a particular subject possesses certain properties or characteristics. “Identifying” may include, for example, self-identification, self-diagnosis, and diagnosis by a medical professional.

As used herein, “treat,” “treatment,” or “treating,” refers to administering or providing a composition for prophylactic and/or therapeutic purposes.

As used herein, the terms “prophylactic treatment,” “prevent,” or “preventing,” refers to treating a subject who does not yet exhibit symptoms of a disease or condition, but who is susceptible to, or otherwise at risk of, a particular disease or condition, whereby the treatment reduces the likelihood that the patient will develop the disease or condition.

As used in the claims below and throughout this disclosure, the phrase “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and can or cannot be present depending upon whether or not they affect the activity or action of the listed elements.

In some embodiments, the compositions disclosed herein are provided to the subject orally. In other embodiments, the compositions disclosed herein are provided by any other convenient route, for example, by intravenous infusion or bolus injection, by absorption through epithelial or mucocutaneous linings (e.g., oral mucosa, rectal and intestinal mucosa, etc.) and can be administered together with another biologically active agent. Administration can be systemic or local. Various delivery systems useful in the methods disclosed herein include for example, encapsulation in liposomes, microparticles, microcapsules, capsules, etc., and can be used to administer a compound of the invention. In certain embodiments, more than one composition disclosed herein is administered to an individual.

Other modes of administration useful in the methods include but are not limited to intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin. The preferred mode of administration is left to the discretion of the professional, and will depend, in-part, upon the site of the condition to be treated. In most instances, administration will result in the release of the compositions disclosed herein into the bloodstream.

In specific embodiments, it can be desirable to administer one or more compositions disclosed herein locally to the area in need of treatment. This can be achieved, for example, and not by way of limitation, by local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. In one embodiment, administration can be by direct injection at the site (or former site) of an atherosclerotic plaque tissue

In certain embodiments, for example, for the treatment of Alzheimer's disease, it may be desirable to introduce one or more compositions disclosed herein into the central nervous system by any suitable route, including intraventricular, intrathecal or epidural injection. Intraventricular injection may be facilitated by an intraventricular catheter, for example, attached to a reservoir, such as an Ommaya reservoir.

Pulmonary administration can also be employed, e.g., by use of an inhaler or nebulizer, and formulation with an aerosolizing agent, or via perfusion in a fluorocarbon or synthetic pulmonary surfactant. In certain embodiments, the compositions disclosed herein can be formulated as a suppository, with traditional binders and vehicles such as triglycerides.

Preferably, the compositions disclosed herein are formulated with a pharmaceutically acceptable vehicle. As used herein, the term “pharmaceutically acceptable” means approved by a regulatory agency of the Federal or a state government or listed in the U.S. Pharmacopeia or other generally recognized pharmacopeia for use in animals, and more particularly in humans. The term “vehicle” refers to a diluent, adjuvant, excipient, or carrier with which a compound of the invention is administered. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening, lubricating and coloring agents may be used. When administered to a patient, the compositions of the invention and pharmaceutically acceptable vehicles are preferably sterile. Water is a preferred vehicle when the composition is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use.

In some embodiments, the compositions disclosed herein are formulated for oral delivery, for example in the form of tablets, lozenges, aqueous or oily suspensions, granules, powders, emulsions, capsules, syrups, or elixirs. Compounds and compositions described herein for oral delivery can also be formulated in foods and food mixes. Orally administered compositions can contain one or more optionally agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active driving compound are also suitable for orally administered compounds and compositions described herein. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time delay material such as glycerol monostearate or glycerol stearate can also be used. Oral compositions can include standard vehicles such as mannitol, lactose, starch, magnesium stearate, sodium saccharine, cellulose, magnesium carbonate, etc. Such vehicles are preferably of pharmaceutical grade.

In some embodiments, the compositions described herein can be in the form of nutraceutical packs not limited to functional foods, beverages, bars, dietary supplements, capsules, powder form or gelatin form, pharmaceutical packs or kits comprising one or more containers filled with one or more compositions disclosed herein. Optionally associated with such container(s) can be a notice in the form prescribed by a governmental agency regulating the manufacture, use or sale of pharmaceuticals or biological products, which notice reflects approval by the agency of manufacture, use or sale for human administration. In a certain embodiment, the nutraceuticals can be in the form of a kit that contains more than one compound described herein.

The compositions disclosed herein can be assayed in vitro and in vivo, for the desired therapeutic or prophylactic activity, prior to use in humans. For example, in vitro assays can be used to determine whether administration of a specific compound described herein or a combination of compositions disclosed herein is preferred for improving symptoms associated with altered glucose metabolism, chromium deficiency, or any other disease or condition disclosed herein. The compositions disclosed herein also be demonstrated to be effective and safe using animal model systems.

In some embodiments, a formulation includes at least about 5% (w/w) of a particular form of CrHis. In some embodiments, a formulation includes at least about 10% (w/w) of a particular form of CrHis. In some embodiments, a formulation includes at least about 15% (w/w) of a particular form of CrHis. In some embodiments, a formulation includes at least about 20% (w/w) of a particular form of CrHis. In some embodiments, a formulation includes at least about 25% (w/w) of a particular form of CrHis. In some embodiments, a formulation includes at least about 30% (w/w) of a particular form of CrHis. In some embodiments, a formulation includes at least about 33% (w/w) of a particular form of CrHis. In some embodiments, a formulation includes at least about 50% (w/w) of a particular form of CrHis. In some embodiments, a formulation includes at least about 60% (w/w) of a particular form of CrHis. In some embodiments, a formulation includes at least about 70% (w/w) of a particular form of CrHis. In some embodiments, a formulation includes at least about 80% (w/w) of a particular form of CrHis. In some embodiments, a formulation includes at least about 90% (w/w) of a particular form of CrHis. In some embodiments, a formulation includes at least about 95% (w/w) of a particular form of CrHis. In some embodiments, a formulation includes at least about 96% (w/w) of a particular form of CrHis. In some embodiments, a formulation includes at least about 97% (w/w) of a particular form of CrHis. In some embodiments, a formulation includes at least about 98% (w/w) of a particular form of CrHis. In some embodiments, a formulation includes at least about 99% (w/w) of a particular form of CrHis.

In some embodiments, a formulation includes at least about 50% (w/w) of one particular form of CrHis and at least about 50% (w/w) of another particular form of CrHis. For example, in some embodiments, the formulation includes about 50% (w/w) of Isomer 1 and about 50% (w/w) of Isomer 2. In some embodiments, a formulation includes at least about 60% (w/w) of one particular form of CrHis and at least about 40% (w/w) of another particular form of CrHis. For example, in some embodiments, the formulation includes about 60% (w/w) of Isomer 1 and about 40% (w/w) of Isomer 2. In some embodiments, a formulation includes at least about 70% (w/w) of one particular form of CrHis and at least about 30% (w/w) of another particular form of CrHis. For example, in some embodiments, the formulation includes about 70% (w/w) of Isomer 1 and about 30% (w/w) of Isomer 2. In some embodiments, a formulation includes at least about 80% (w/w) of one particular form of CrHis and at least about 20% (w/w) of another particular form of CrHis. For example, in some embodiments, the formulation includes about 80% (w/w) of Isomer 1 and about 20% (w/w) of Isomer 2. In some embodiments, a formulation includes at least about 90% (w/w) of one particular form of CrHis and at least about 10% (w/w) of another particular form of CrHis. For example, in some embodiments, the formulation includes about 90% (w/w) of Isomer 1 and about 10% (w/w) of Isomer 2.

In some embodiments, a formulation includes at least about 20% (w/w) of one particular form of CrHis and at least about 20% (w/w) of another particular form of CrHis, and at least about 20% (w/w) of another particular form of CrHis. For example, in some embodiments, the formulation includes about 20% (w/w) of Isomer 1, about 20% (w/w) of Isomer 2, and about 20% (w/w) of Isomer 3. In some embodiments, a formulation includes at least about 25% (w/w) of one particular form of CrHis and at least about 25% (w/w) of another particular form of CrHis, and at least about 25% (w/w) of another particular form of CrHis. For example, in some embodiments, the formulation includes about 25% (w/w) of Isomer 1, about 25% (w/w) of Isomer 2, and about 25% (w/w) of Isomer 3. In some embodiments, a formulation includes at least about 30% (w/w) of one particular form of CrHis and at least about 30% (w/w) of another particular form of CrHis, and at least about 30% (w/w) of another particular form of CrHis. For example, in some embodiments, the formulation includes about 30% (w/w) of Isomer 1, about 30% (w/w) of Isomer 2, and about 30% (w/w) of Isomer 3. In some embodiments, a formulation includes at least about 33% (w/w) of one particular form of CrHis and at least about 33% (w/w) of another particular form of CrHis, and at least about 33% (w/w) of another particular form of CrHis. For example, in some embodiments, the formulation includes about 33% (w/w) of Isomer 1, about 33% (w/w) of Isomer 2, and about 33% (w/w) of Isomer 3.

In some embodiments, the formulation is substantially free of free histidine, or a salt thereof. In some embodiments, the formulation includes at most about 30% (w/w) free histidine, or a salt thereof. In some embodiments, the formulation includes at most about 25% (w/w) free histidine, or a salt thereof. In some embodiments, the formulation includes at most about 20% (w/w) free histidine, or a salt thereof. In some embodiments, the formulation includes at most about 15% (w/w) free histidine, or a salt thereof. In some embodiments, the formulation includes at most about 12% (w/w) free histidine, or a salt thereof. In some embodiments, the formulation includes at most about 10% (w/w) free histidine, or a salt thereof. In some embodiments, the formulation includes at most about 8% (w/w) free histidine, or a salt thereof. In some embodiments, the formulation includes at most about 5% (w/w) free histidine, or a salt thereof. In some embodiments, the formulation includes at most about 4% (w/w) free histidine, or a salt thereof. In some embodiments, the formulation includes at most about 3% (w/w) free histidine, or a salt thereof. In some embodiments, the formulation includes at most about 2% (w/w) free histidine, or a salt thereof. In some embodiments, the formulation includes at most about 1% (w/w) free histidine, or a salt thereof.

Some embodiments provide a mixture of two isomers of CrHis, wherein the mixture is substantially free of other CrHis isomers. In some embodiments, the ratio of the two CrHis isomers is from about 1:100 to about 100:1. Some embodiments provide a mixture of three isomers of CrHis, wherein the mixture is substantially free of other CrHis isomers. In some embodiments, the ratio of the three CrHis isomers is from about 1:1:100 to about 1:100:1 to about 100:1:1. Some embodiments provide a mixture of two isomers of CrHis, wherein the mixture is substantially free of other CrHis isomers. In some embodiments, the ratio of the two CrHis isomers is from about 1:10 to about 10:1. Some embodiments provide a mixture of three isomers of CrHis, wherein the mixture is substantially free of other CrHis isomers. In some embodiments, the ratio of the three CrHis isomers is from about 1:1:10 to about 1:10:1 to about 10:1:1. In some embodiments, the ratio of the three CrHis isomers is about 1:1:1.

Some embodiments include forms of CrHis complexes and/or combinations of forms of CrHis complexes that are concentrated, purified, and/or substantially purified. Pure, as used herein, refers to forms of CrHis compounds that are free (or at least primarily free) of other forms of CrHis compounds. Substantially pure forms of CrHis compounds, as used herein, refers to forms of CrHis compounds that have substantially more of a particular form than another form and/or substantially more of a certain form than those CrHis compounds made by previously disclosed techniques.

In some embodiments, a formulation includes at least about 50% (w/w) of a particular form of CrPic. In some embodiments, a formulation includes at least about 60% (w/w) of a particular form of CrPic. In some embodiments, a formulation includes at least about 70% (w/w) of a particular form of CrPic. In some embodiments, a formulation includes at least about 80% (w/w) of a particular form of CrPic. In some embodiments, a formulation includes at least about 90% (w/w) of a particular form of CrPic. In some embodiments, a formulation includes at least about 95% (w/w) of a particular form of CrPic. In some embodiments, a formulation includes at least about 96% (w/w) of a particular form of CrPic. In some embodiments, a formulation includes at least about 97% (w/w) of a particular form of CrPic. In some embodiments, a formulation includes at least about 98% (w/w) of a particular form of CrPic. In some embodiments, a formulation includes at least about 99% (w/w) of a particular form of CrPic. In some embodiments, a formulation includes at least about 99.5% (w/w) of a particular form of CrPic. In some embodiments, a formulation includes at least about 99.9% (w/w) of a particular form of CrPic.

In some embodiments, a formulation includes at least about 50% (w/w) of one particular form of CrPic and at least about 50% (w/w) of another particular form of CrPic. For example, in some embodiments, the formulation includes about 50% (w/w) of Isomer 1 and about 50% (w/w) of Isomer 2. In some embodiments, a formulation includes at least about 60% (w/w) of one particular form of CrPic and at least about 40% (w/w) of another particular form of CrPic. For example, in some embodiments, the formulation includes about 60% (w/w) of Isomer 1 and about 40% (w/w) of Isomer 2. In some embodiments, a formulation includes at least about 70% (w/w) of one particular form of CrPic and at least about 30% (w/w) of another particular form of CrPic. For example, in some embodiments, the formulation includes about 70% (w/w) of Isomer 1 and about 30% (w/w) of Isomer 2. In some embodiments, a formulation includes at least about 80% (w/w) of one particular form of CrPic and at least about 20% (w/w) of another particular form of CrPic. For example, in some embodiments, the formulation includes about 80% (w/w) of Isomer 1 and about 20% (w/w) of Isomer 2. In some embodiments, a formulation includes at least about 90% (w/w) of one particular form of CrPic and at least about 10% (w/w) of another particular form of CrPic. For example, in some embodiments, the formulation includes about 90% (w/w) of Isomer 1 and about 10% (w/w) of Isomer 2. In some embodiments, a formulation includes at least about 99% (w/w) of one particular form of CrPic and at least about 1% (w/w) of another particular form of CrPic. For example, in some embodiments, the formulation includes about 99% (w/w) of Isomer 1 and about 1% (w/w) of Isomer 2. In some embodiments, a formulation includes at least about 99.5% (w/w) of one particular form of CrPic and at least about 0.5% (w/w) of another particular form of CrPic. For example, in some embodiments, the formulation includes about 99.5% (w/w) of Isomer 1 and about 0.5% (w/w) of Isomer 2. In some embodiments, a formulation includes at least about 99.9% (w/w) of one particular form of CrPic and at least about 0.1% (w/w) of another particular form of CrPic. For example, in some embodiments, the formulation includes about 99.9% (w/w) of Isomer 1 and about 0.1% (w/w) of Isomer 2. In other embodiments, the amount of Isomer 2 can be increased. For example, in some embodiments, the improved formulations described herein include >2% (w/w) CrPic Isomer 2.

In some embodiments, the formulation is substantially free of free picolinic acid, or a salt thereof. In some embodiments, the formulation includes at most about 40% (w/w) free picolinic acid, or a salt thereof. In some embodiments, the formulation includes at most about 35% (w/w) free picolinic acid, or a salt thereof. In some embodiments, the formulation includes at most about 30% (w/w) free picolinic acid, or a salt thereof. In some embodiments, the formulation includes at most about 25% (w/w) free picolinic acid, or a salt thereof. In some embodiments, the formulation includes at most about 20% (w/w) free picolinic acid, or a salt thereof. In some embodiments, the formulation includes at most about 15% (w/w) free picolinic acid, or a salt thereof. In some embodiments, the formulation includes at most about 10% (w/w) free picolinic acid, or a salt thereof. In some embodiments, the formulation includes at most about 5% (w/w) free picolinic acid, or a salt thereof. In some embodiments, the formulation includes at most about 4% (w/w) free picolinic acid, or a salt thereof. In some embodiments, the formulation includes at most about 3% (w/w) free picolinic acid, or a salt thereof. In some embodiments, the formulation includes at most about 2% (w/w) free picolinic acid, or a salt thereof. In some embodiments, the formulation includes at most about 1% (w/w) free picolinic acid, or a salt thereof.

Some embodiments provide a mixture of two isomers of CrPic, wherein the mixture is substantially free of other CrPic isomers. In some embodiments, the ratio of the two CrPic isomers is from about 1:100 to about 100:1. Some embodiments provide a mixture of three isomers of CrPic, wherein the mixture is substantially free of other CrPic isomers. Some embodiments provide a mixture of two isomers of CrPic, wherein the mixture is substantially free of other CrPic isomers. In some embodiments, the ratio of the two CrPic isomers is from about 1:10 to about 10:1.

Some embodiments include forms of CrPic complexes that are concentrated, purified, and/or substantially purified. Pure, as used herein, refers to forms of CrPic compounds that are free (or at least primarily free) of other forms of CrPic compounds. Substantially pure forms of CrPic compounds, as used herein, refers to forms of CrPic compounds that have substantially more of a particular form than another form and/or substantially more of a certain form than those CrPic compounds made by previously disclosed techniques.

In some embodiments, the formulation comprises a mixture of CrHis isomers and CrPic isomers. In some embodiments, the formulation comprises at least about 1% (w/w) CrPic. In some embodiments, the formulation comprises at least about 5% (w/w) CrPic. In some embodiments, the formulation comprises at least about 10% (w/w) CrPic. In some embodiments, the formulation comprises at least about 20% (w/w) CrPic. In some embodiments, the formulation comprises at least about 30% (w/w) CrPic. In some embodiments, the formulation comprises at least about 40% (w/w) CrPic. In some embodiments, the formulation comprises at least about 50% (w/w) CrPic. In some embodiments, the CrHis in the formulation comprises less than about 15% CrHis isomer 1; more than about 12% CrHis isomer 2; more than about 22% CrHis isomer 3; and less than about 35% free histidine. In some embodiments, the CrHis in the formulation comprises less than about 12% CrHis isomer 1; more than about 15% CrHis isomer 2; more than about 30% CrHis isomer 3; and less than about 25% free histidine. In some embodiments, the CrHis in the formulation comprises less than about 10% CrHis isomer 1; more than about 18% CrHis isomer 2; more than about 20% CrHis isomer 3; and less than about 15% free histidine. In some embodiments, the CrHis in the formulation comprises about 8% CrHis isomer 1; about 20% CrHis isomer 2; about 50% CrHis isomer 3; and less than about 8% free histidine.

While the present invention has been described in some detail for purposes of clarity and understanding, one will appreciate that various changes in form and detail can be made without departing from the true scope of the invention.

### Example 1

### Structural Identity of Certain Forms of CrHis Compounds

The following study was undertaken to determine the structural identity of certain chromatographically separable components of interest which were detectable in the HPLC analysis of the chromium histidinate drug substances. This study describes the development of HPLC-UV methods for the assay and the potential impurity determination of chromium histidinate. During the development of the HPLC-UV methods, the chromium histidinate drug substance was found to contain histidine plus three major separable peaks of unknown origin. The identity of the three chromium histidinate components was investigated by LC/MS analysis.

The following materials were utilized:
Agilent 1100 Series LC/MS System consisting of the following components:
   HPLC Binary Pumps
   Degasser Unit
   Column Oven Compartment
   Autosampler Unit
   Diode Array Detector
   MSD Single-Quadrapole Mass. Selective Detector
Computer with Chemstation® Software as data collection system
Waters Atlantis HILIC Silica HPLC Column, 4.6 mm×150 mm, 3μ Particle Size
AND HM-202 Analytical Balance
Fisher AR25 pH Meter
Acetonitrile, Fisher, LC/MS Optima grade
Water Fisher, LC/MS Optima grade
Ammonium Acetate, Baker, Baker-analyzed HPLC Reagent grade
Glacial Acetic Acid, Fisher, ACS grade
Chromium Histidinate (from Nutrition 21) Lot#C12711

Chromium histidinate solution was prepared using 50:50 acetonitrile/water as diluent at a concentration of 10 mg/ml. The HPLC conditions developed in the previous HPLC method development work were transferable directly to LC/MS without adaptation, as the mobile phase components consist of volatile organic salts, solvents and water. The HPLC conditions are shown in Table 1.

TABLE 1
Chromium Histidinate HPLC Conditions

| | |
|---|---|
| Analytical Column: | Waters Atlantis HILIC Silica, |
| | 4.6 × 150 mm, 3 um PS |
| Mobile Phase: | 80:20 Acetonitrile/50 mM |
| | Ammonium Acetate, pH 5.3 |
| Flow Rate: | 1 ml/min |
| HPLC run type | Isocratic |
| (isocratic/gradient): | |
| Detection: | UV |
| Wavelength: | 214 nm |
| Column Temperature: | 30 C |
| Injection Volume: | 30 ul |
| Run Time: | 60 min |
| Sample cooler temperature | Ambient |

The HPLC flow was analyzed by MS using the API-ES (atmospheric pressure ionization—electrospray) in the positive mode using a scan range of m/z 100-1500. The MS parameters for the analysis are shown in Table 2.

TABLE 2
Chromium Histidinate MS Parameters

| | |
|---|---|
| Ion Source: | API-ES |
| Scan Range: | 100-1500 |
| Fragmentor Voltage: | 70 eV |
| Gain Setting | 1.00 |
| Threshold: | 0 |
| Step Size: | 0.1 |
| Drying Gas Temp: | 350° C. |
| Drying Gas Flow: | 12 L/min |
| Nebulizer Pressure: | 35 psig |
| Vaporizer Temperature: | 350° C. |
| Capillary Voltage | 3000 V |
| (Positive): | |
| Capillary Voltage | 3000 V |
| (Negative): | |

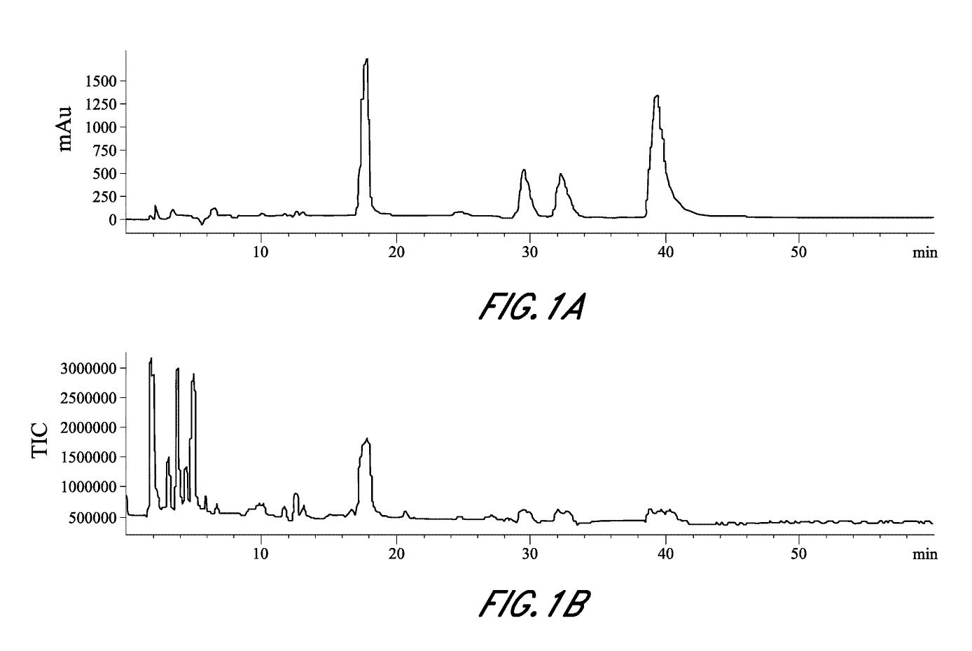

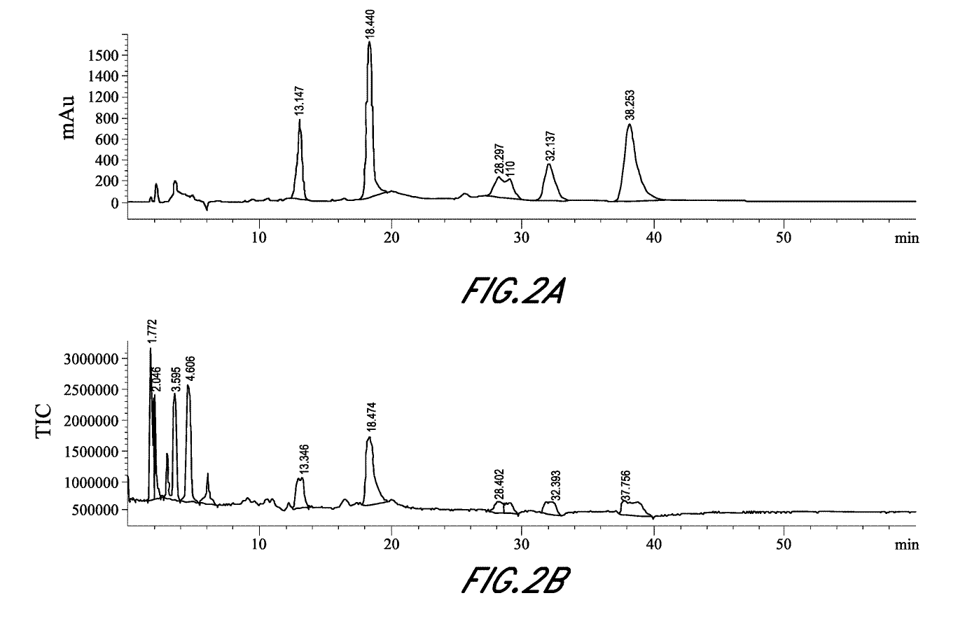

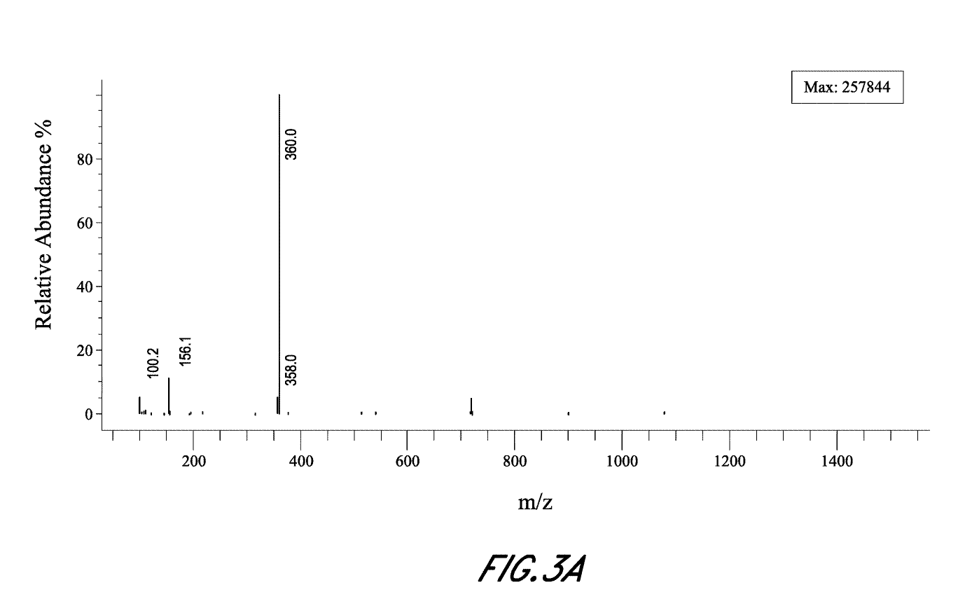

The resulting chromatograms for the UV (214 nm) trace and the MS TIC are shown in FIGS. 1A-1B. The three late eluting peaks of interest were readily identifiable in the UV trace, labeled as peaks 1, 2 and 3 in FIG. 1A. Each peak showed a corresponding peak in the MS TIC trace (see, FIGS. 1A-1B & FIGS. 2A-2B), indicating that the compound was detectable by MS using the chosen ionization technique. It was notable that a multitude of peaks were observed in the MS TIC during the first seven minutes of elution which did not present as major components in the UV trace. The finding suggests that other components are present which are not readily detectable by UV absorbance measurement.

It should be noted that the freshly prepared solution of chromium histidinate resulted in a slightly different chromatographic profile than that shown in FIGS. 1A-1B. The profile of the freshly prepared solution is shown in FIGS. 2A-2B. The profile shown in FIGS. 1A-1B was from a solution which is approximately one week old. The investigation focused on the three peaks shown in FIGS. 1A-1B.

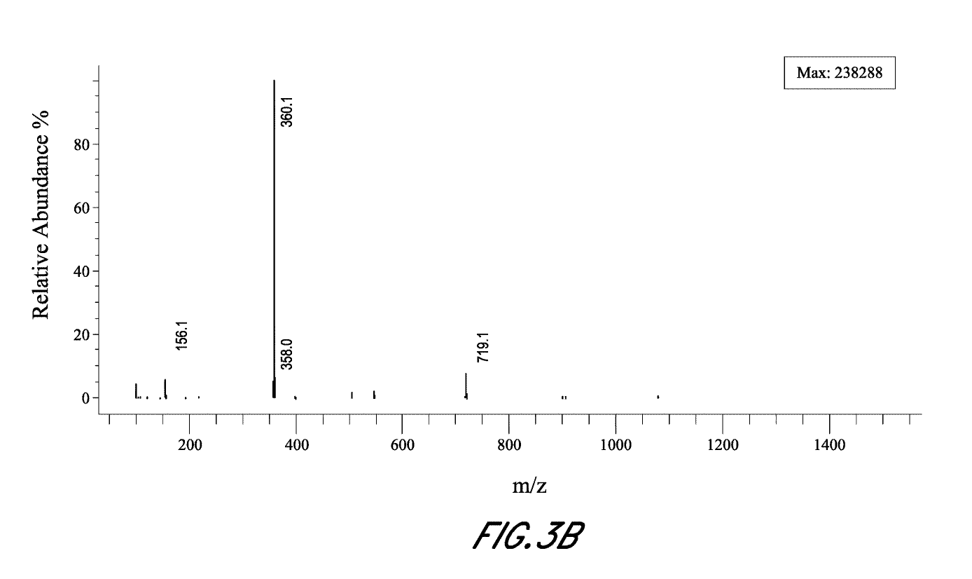

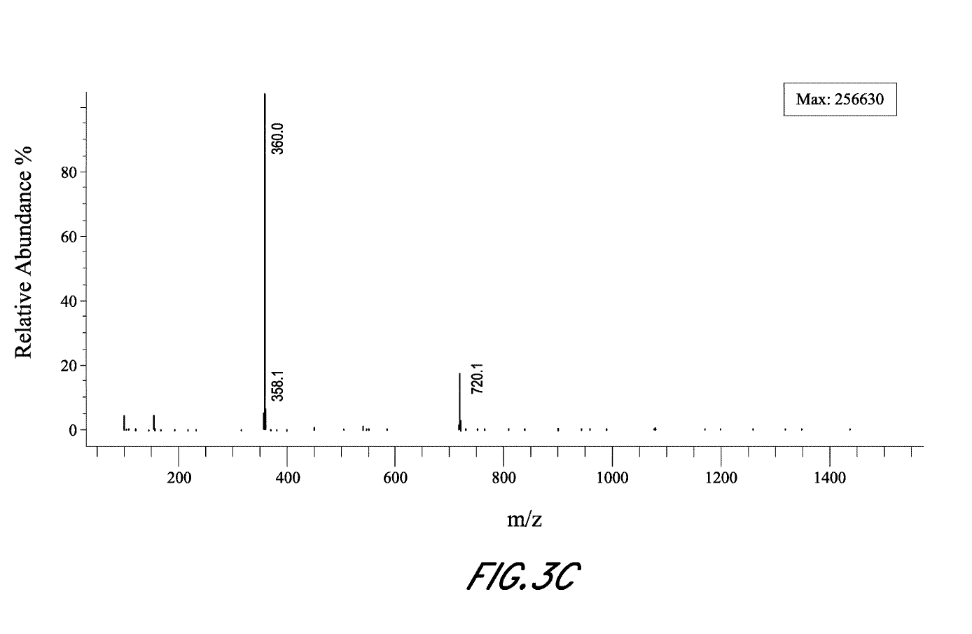

Mass spectra were generated from each of the three peaks detected in the MS TIC. The spectrum from each peak from FIGS. 1A-1B and is shown in FIGS. 3A-3C. The spectra of each peak were found to be very similar—each having a base peak at m/z 360 and having highest major cluster at m/z 719. Furthermore, the spectra each showed a peak at m/z˜156 and in each spectra, and the peak clusters at m/z 360 and m/z 719 exhibited a characteristic isotopic abundance pattern in each case (see FIGS. 3A-3B). The fragment at m/z 156 suggests the presence of histidine in the compound as the histidine (M+H)+ ion has m/z 156. The isotopic abundance pattern seen in the peak clusters at m/z 360 and m/z 719 are consistent with the expected natural isotopic abundance of chromium (see Table 3). Therefore it was deduced that each peak has chromium and histidinate components, and each peak likely has a related structure; most likely the three are structural isomers described below. The spectral peaks at m/z 360 and m/z 719 are consistent with the (M+2H)2+ and the (M+H)+ ions, respectively of a neutral mass of m/z 718.

TABLE 3
Natural Abundance of the Four Chromium Isotopes

| Chromium Isotope | % Abundance in Nature |
|---|---|
| ⁵⁰Cr | 4% |
| ⁵²Cr | 84% |
| ⁵³Cr | 10% |
| ⁵⁴Cr | 2% |

See Atomic weights of the elements, IUPAC Technical Report (2000).

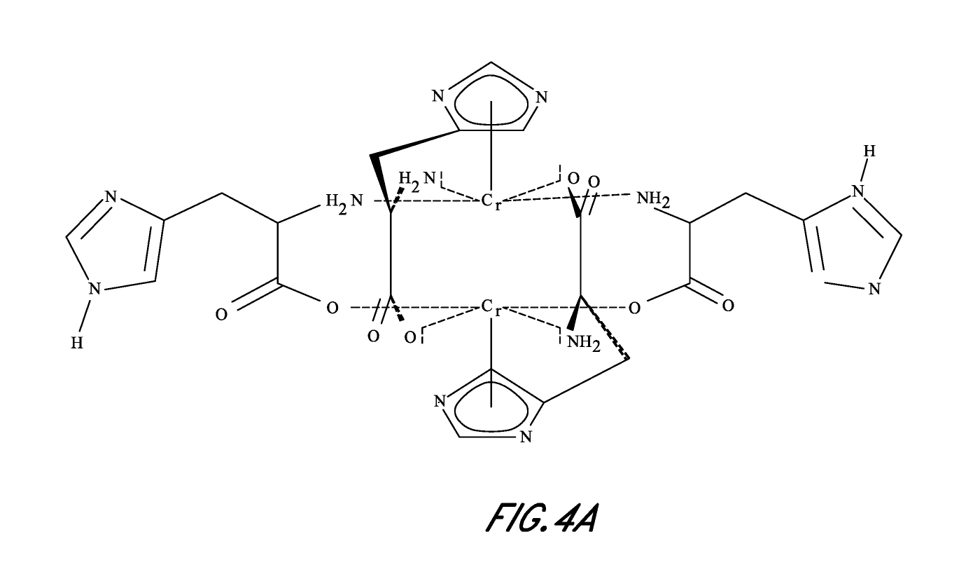

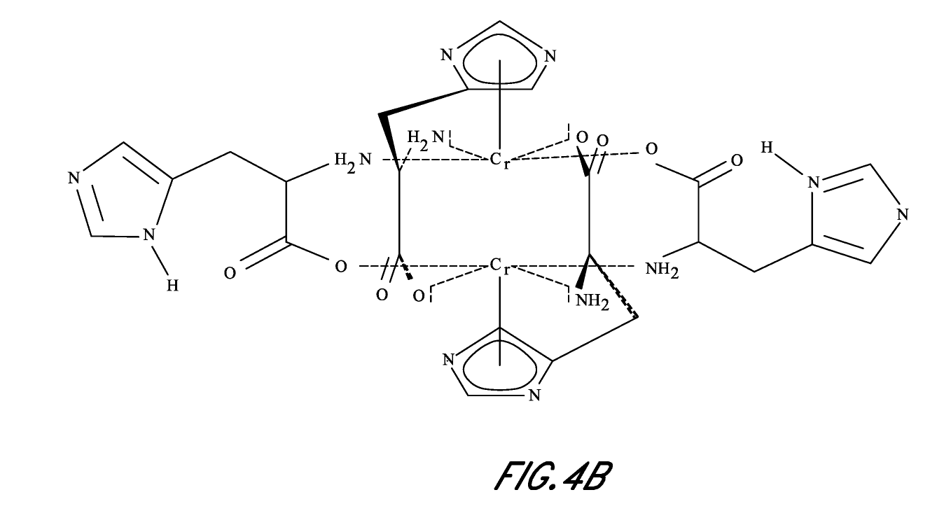

Three structures were proposed for complexes of chromium (III) and histidinate which are consistent with the molecular weight of 718, and are also consistent with the expected coordination chemistry of chromium. The three structures are presented in FIGS. 4A-4C. The three structures having the molecular formula Cr₂(Histidinate)⁴ represent three possible arrangements of four histidinate molecules around two chromium atoms. The possibility of three chemically different structural isomers with this formula agrees with the finding of three separable peaks on the HPLC.

Each of the three structures proposed for chromium histidinate feature two chromium atoms, each coordinated by either the lone pair of the nitrogen of the amino group or a lone pair of the negative charge-bearing oxygen of the carboxyl group, as well as a negative charge bearing arene ligand of the imidazole moiety. The arrangement of these ligands around the two chromium atoms at the core of the complex dictates the structure of the complex.

Isomer 3 in FIG. 4C features the carboxyl groups on opposed histidine residues around an individual chromium atom (carboxyl groups are at 180 degree angles from one another). There is the potential for hydrogen bonding between amino groups of histidinate and the carboxyl groups of neighboring histidinate ligands (histidinate ligands at 90 degree angles relative to one another) in this structure. The opposed disposition of the negatively charged carboxyl groups in this structure give it the most symmetrical charge distribution of the three structures, suggesting it may be the most energetically favorable configuration. The potential for intramolecular hydrogen bonding in this structure theoretically imparts the structure more cohesive stability, thus less fragmentation is to be expected in MS detection.

The predicted properties of Isomer 3 correspond to the observations on the largest of the three peaks, observed at ˜39.5 minutes. This structure is the expected major product of the reaction of chromium (III) with histidinate, thus would be expected to result in the largest peak on HPLC (assuming similar extinction coefficients of the three compounds). The predicted hydrogen bonding behavior is expected to result in a larger molecular ion at m/z=719 and smaller histidine fragment at m/z=156. This prediction conforms to the observed behavior in the spectrum of the peak at ˜39.5 minutes shown in FIG. 3C.

Likewise, Isomer 2 shown in FIG. 4B, features carboxyl groups on adjacent histidine residues coordinating single chromium atom. In this configuration, charge distribution is less symmetrical than in Isomer 3—thus the structure is less energetically favorable. Furthermore, the amino groups are capable of hydrogen bonding with the neighboring carboxyl group, but not with its other neighboring group, another amino group. Reduced potential for hydrogen bonding behavior exists compared to Isomer 3 and Isomer 2 therefore is predicted to have a comparatively smaller molecular ion due to a higher propensity to fragment.

Isomer 1 shown in FIG. 4A, features the least energetically stable configuration due to the uneven distribution of charge. In Isomer 1 three of the four carboxyl groups are coordinating one chromium atom. Isomer 1 has the least potential for hydrogen bonding among adjacent histidinate ligands, thus it is expected to have the smallest molecular ion signal and the greatest fragmentation. The expected properties of Isomer 1 correspond to the finding that the peak eluting at ˜29.5 minutes gave the spectrum which showed the lowest magnitude of the molecular ion and the highest magnitude histidine fragment. Isomer 2, having intermediate properties, is assigned to the peak at ˜32.5 min. Peaks 1, 2 and 3 as labeled in FIGS. 1A-1B are tentatively assigned to Isomers 1, 2 and 3 respectively as shown in FIGS. 4A-4C.

### Example 2

### Structural Identity of Certain Forms of CrPic Compounds

The following study was undertaken to determine the structural identity of certain chromatographically separable components of interest which were detectable in the HPLC analysis of the chromium picolinate drug substances. This study describes the development of HPLC-UV methods for the assay and the potential impurity determination of chromium picolinate. During the development of the HPLC-UV methods, the chromium picolinate drug substance was found to contain picolinate plus two major separable peaks of unknown origin. The identity of the two chromium picolinate components was investigated by LC/MS analysis.

The following materials were utilized:
Agilent 1100 Series LC/MS System consisting of the following components:
   HPLC Binary Pumps
   Degasser Unit
   Column Oven Compartment
   Autosampler Unit
   Diode Array Detector
   MSD Single-Quadrapole Mass. Selective Detector
Computer with Chemstation® Software as data collection system
Waters Atlantis HILIC Silica HPLC Column, 4.6 mm×150 mm, 3μ Particle Size
AND HM-202 Analytical Balance
Fisher AR25 pH Meter
Acetonitrile, Fisher, LC/MS Optima grade
Water Fisher, LC/MS Optima grade
Ammonium Acetate, Baker, Baker-analyzed HPLC Reagent grade
Glacial Acetic Acid, Fisher, ACS grade
Chromax Chromium Picolinate (from Nutrition 21) 12.18% Chromium (anhydrous), Lot#N21ST02

Chromium picolinate solution was prepared using 50:50 acetonitrile/water as a diluent at a concentration of 10 mg/mL. The HPLC conditions developed in the previous HPLC method development work were transferable directly to LC/MS without adaptation, as the mobile phase components consist of volatile organic salts, solvents and water. The HPLC conditions are shown in Table 4.

TABLE 4
Chromium Picolinate HPLC Conditions

| | |
|---|---|
| Analytical Colum | Waters Atlantis HILIC Silica, |
| | 4.6 × 150 mm, 3 um PS |
| Mobile Phase: | 95:5 Acetonitrile/50 mM Ammonium Acetate |
| Flow Rate: | 1 ml/min |
| HPLC run type | Isocratic |
| (isocratic/gradient): | |
| Detection: | UV |
| Wavelength: | 265 nm |
| Column Temperature: | 30 C |
| Injection Volume: | 10 ul |
| Run Time: | ~12 min |
| Sample cooler temperature | Ambient |

The HPLC flow was analyzed by MS using the APCI (atmospheric pressure chemical ionization) in the positive mode using a scan range of m/z 100-1500. The MS parameters for the analysis are shown in Table 5.

TABLE 5
Chromium Picolinate MS Parameters

| | |
|---|---|
| Ion Source: | APCI |
| Scan Range: | 100-1500 |
| Fragmentor Voltage: | 70 eV |
| Gain Setting | 1.00 |
| Threshold: | 0 |
| Step Size: | 0.1 |
| Drying Gas Temp: | 350° C. |
| Drying Gas Flow: | 12 L/min |
| Nebulizer Pressure: | 35 psig |
| Vaporizer Temperature: | 450° C. |
| Capillary Voltage | 3000 V |
| (Positive): | |
| Capillary Voltage | 3000 V |
| (Negative): | |
| Corona Discharge | 4.0 μA |
| (Positive): | |
| Corona Discharge | 15.0 μA |
| (Negative): | |

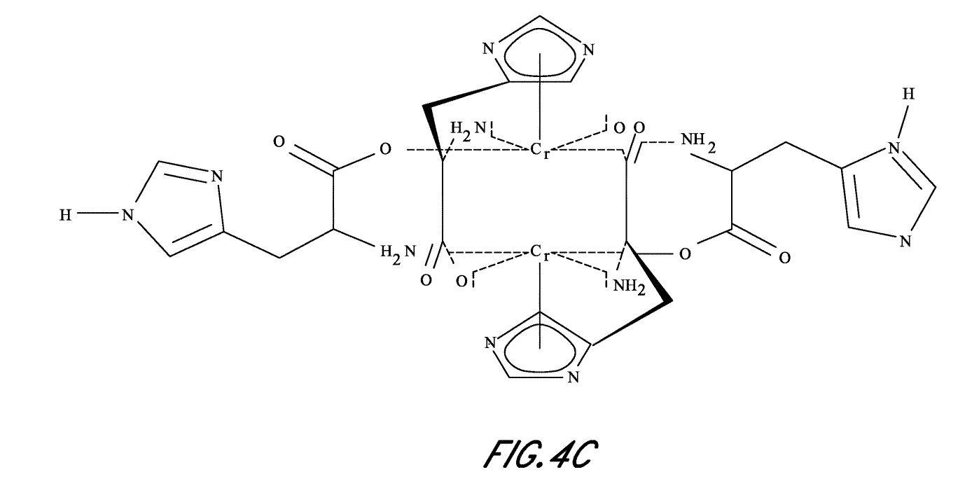

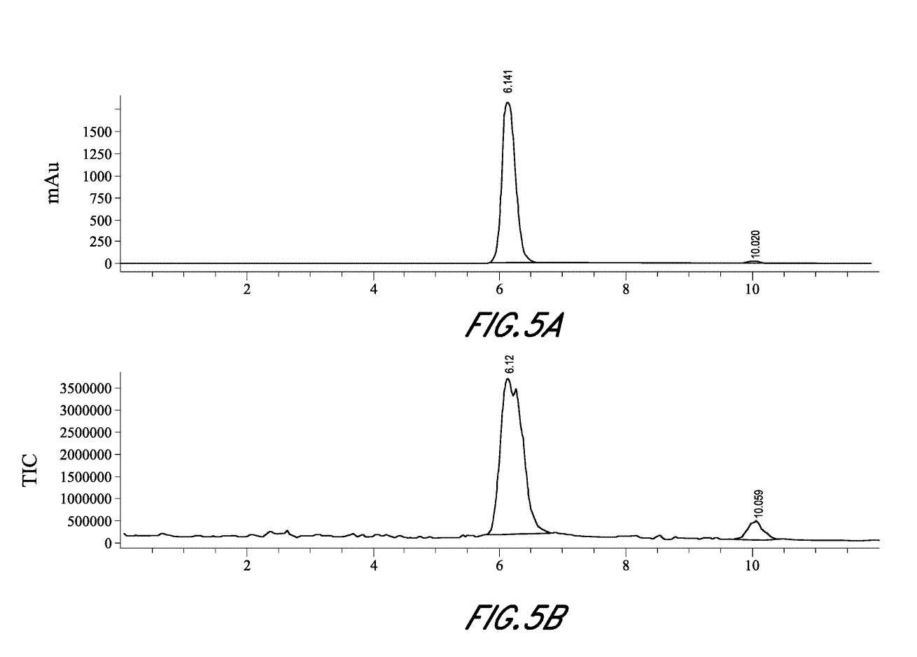

The resulting chromatograms for the UV (265 nm) trace and the MS TIC are shown in FIGS. 5A-5B, respectively. The impurity peak which was previously observed during previous method development work was readily identifiable in the UV trace, labeled as such in FIGS. 5A-5B. The impurity peak showed a corresponding peak in the MS TIC trace, indicating that the compound was detectable by MS using the chosen ionization technique. A mass spectrum was generated from the chromium picolinate impurity peak at ˜10.1 minutes. The spectrum is shown in FIG. 6.

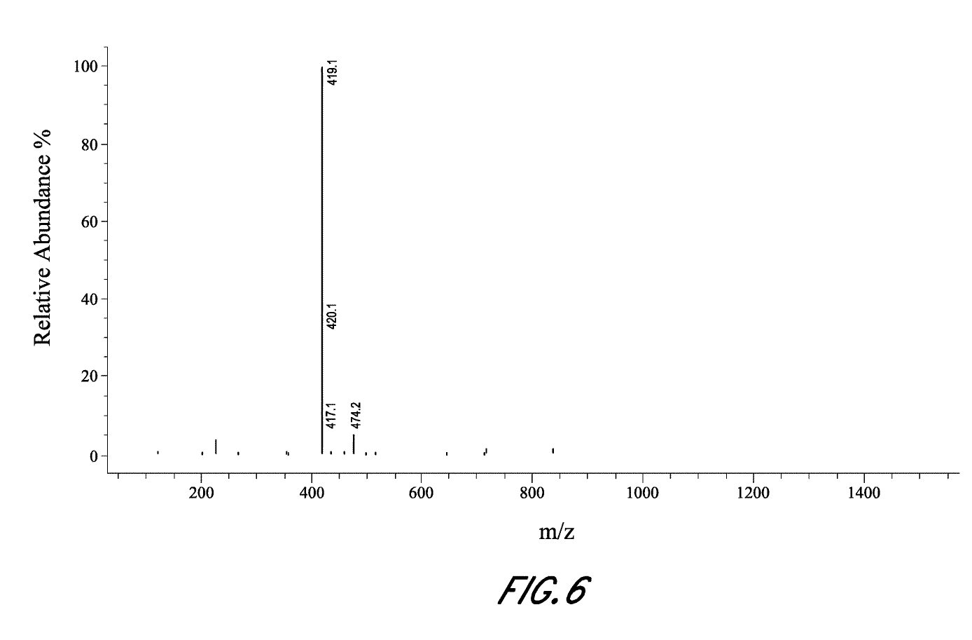

The spectrum of the impurity peak was shown to have the base peak at m/z 419, corresponding to the (M+H)+ ion of chromium (III) picolinate, MW 418. Because the impurity peak apparently shares the molecular weight of the main compound, yet is chemically separable by chromatography, it was deduced that the impurity is likely a structural isomer of chromium picolinate. Two possible configurations of chromium (III) coordinated by three picolinate molecules are shown in FIGS. 7A-7B.

CrPic Isomer 1 (FIG. 7A) and CrPic Isomer 2 (FIG. 7B) both feature a chromium atom coordinated by the pyridine nitrogen lone pair and a lone pair of the negative charge-bearing oxygen in the carboxyl group.

In CrPic Isomer 2 (FIG. 7B), each nitrogen atom is opposite an oxygen (180 degrees from an oxygen atom) and vice versa, whereas in CrPic Isomer 1 (FIG. 7A), only one nitrogen is opposite an oxygen atom, and the other two nitrogens sit opposite one another. CrPic Isomer 2 is thought to be less energetically favorable than CrPic Isomer 1 because steric hindrance of the bulky pyridine rings may inhibit the formation of this structure and also because the charge distribution is unfavorable. The pyridine rings as well as the three negatively charged oxygen atoms sit 90 degrees from each other. In contrast, CrPic Isomer 1 features the pyridine moieties as well as the negatively charged carboxylate groups at 90 degree and at 180 degree angles, thereby spreading the bulky aromatic rings and the negative charges over a larger space. By this logic, the impurity is tentatively assigned CrPic Isomer 2, as it fits the profile of a minor product of the reaction of chromium (III) with picolinate, whereas CrPic Isomer 1 is tentatively assigned to the main peak, thought to be the major product of the reaction. The order of elution also supports this argument, as CrPic Isomer 2 has greater polarity, thus is expected to have stronger retention in HILIC mode HPLC.

### Example 3

### Improved Methods of Making CrHis

Chromium histidinate was first made according to the method recited in Example 1 of U.S. Pat. No. 6,689,383. Briefly, three-fold molar excess of histidine is added slowly to chromic acetate or chromic chloride in an aqueous solution at 80° C. The solution is then heated an additional 30 min, cooled to approximately room temperature, and the pH adjusted to pH 5 to 5.5 with concentrated ammonium hydroxide. After cooling, sample can be freeze-dried and used as a nutrient supplement. Other amino acids may also be included in the formulation, but at least one molar equivalent of histidine per mole of chromium must be present. This preparation was compared to chromium histidinate according to the method described below in Table 6 and other similar techniques described in Tables 7-9.

TABLE 6

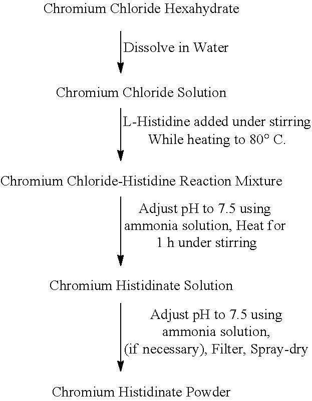

The compositions described above were analyzed for the amount of each chromium isomer contained therein. The various percentages of each isomer according to various methods of making are shown in Tables 5-7. As shown, the method of making chromium histidinate by the methods disclosed herein permits selection of the percentage of each of CrHis Isomer 1, CrHis Isomer 2, and CrHis Isomer 3, in contrast to the prior method. For example, as shown in Table 7, adding a 3× Molar Excess of Histidine and adjusting the pH to 7.5 prior to heating for one hour resulted in a greater percentage of Isomers 1 and 2. This decreases the need for additional purification steps to arrive at CrHis compositions having enriched or depleted amounts of specific CrHis isomers. Additional studies will be performed to further optimize the relative abundance of each isomer. Enriched compositions of each isomer can be further purified by preparative HPLC, and combined to reach desired formulations. It is believed that certain mixtures of CrHis isomers possess advantageous properties over prior art CrHis mixtures, as discussed herein. As shown in Tables 7-9, at least the amount of excess histidine, pH during heating, and final pH all effect the relative amounts of the respective isomers that are present. In some implementations, it was found that more amounts of excess histidine and a neutral pH resulted in greater amounts of the chromium histidine complexes. In some aspects, after formation of the chromium histidine complexes, the amount remaining free histidine is completely or substantially removed. Removal of free histidine results in higher percentages of chromium histidine complexes.

TABLE 7

| | | | | % Total | % | % Other |
|---|---|---|---|---|---|---|
| Desc. | CHC1 % | CHC2 % | CHC3 % | CHCs | Histidine | Peaks |
| Chromium Histidinate, Kelatron Lot | 13.4 | 11.3 | 37.5 | 62.2 | 33.4 | 4.4 |
| Z0805216 | | | | | | |
| Chromium Histidinate Formulated | 20.6 | 15.8 | 30.6 | 67.0 | 32.2 | 0.7 |
| Solution, 3x Molar Excess of Histidine, pH | | | | | | |
| Adjustment to 7.5 prior to Heating 1 h | | | | | | |
| Chromium Histidinate Formulated | 20.4 | 16.0 | 30.7 | 67.1 | 32.3 | 0.6 |
| Solution, 3x Molar Excess of Histidine, pH | | | | | | |
| Adjustment to 7.5 prior to Heating 1 h | | | | | | |
| Chromium Histidinate Formulated | 8.2 | 5.8 | 16.6 | 30.6 | 61.9 | 7.6 |
| Solution, 2x Molar Excess of Histidine, | | | | | | |
| Final pH 7.5 | | | | | | |
| Chromium Histidinate Formulated | 4.9 | 6.7 | 17.3 | 28.9 | 63.5 | 7.5 |
| Solution, 2x Molar Excess of Histidine, | | | | | | |
| Final pH 8.5 | | | | | | |

TABLE 8
Summary Table - Area % HPLC Data - Comparison of 2x Molar
Equivalents of Histidine Adjusted to Final pH 5.25 vs. 7.5

| | | | | % Total | % | % Other |
|---|---|---|---|---|---|---|
| Desc. | CHC1 % | CHC2 % | CHC3 % | CHCs | Histidine | Peaks |
| Chromium Histidinate Formulated | 13.2 | 5.3 | 10.4 | 28.9 | 66.6 | 4.5 |
| Solution, 2x Molar Excess of Histidine, | | | | | | |
| Final pH 5.25 | | | | | | |
| Chromium Histidinate Formulated | 8.2 | 5.8 | 16.6 | 30.6 | 61.9 | 7.6 |
| Solution, 2x Molar Excess of Histidine, | | | | | | |
| Final pH 7.5 | | | | | | |

TABLE 9
Summary Table - Area % HPLC Data - Comparison of Final pH 5.25
(after heating) vs. pH adjustment to 7.5 (before heating)

| | | | | % Total | % | % Other |
|---|---|---|---|---|---|---|
| Desc. | CHC1 % | CHC2 % | CHC3 % | CHCs | Histidine | Peaks |
| Chromium Histidinate Formulated | 9.1 | 9.7 | 30.5 | 49.3 | 45.4 | 5.3 |
| Solution, 3x Molar Excess of Histidine (pH | | | | | | |
| to 5.25 after heating) | | | | | | |
| Chromium Histidinate Formulated | 20.6 | 15.8 | 30.6 | 67.0 | 32.2 | 0.7 |
| Solution, 3x Molar Excess of Histidine, pH | | | | | | |
| Adjustment to 7.5 prior to Heating 1 h | | | | | | |

### Example 4

### Insulin Levels in High-Fat Diet (HFD) Rats

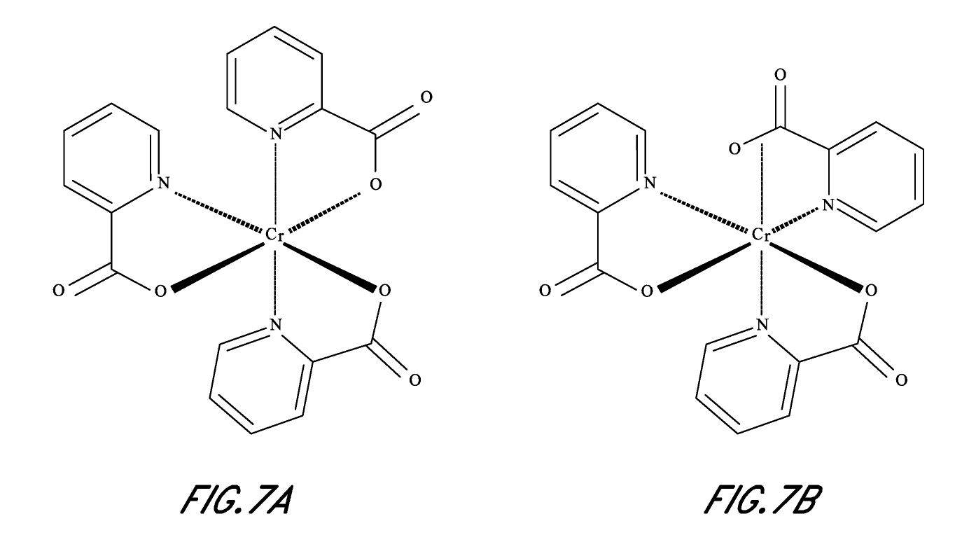

Rats were fed a high-fat diet to induce insulin resistance. Rats were fed the same amount of Cr, but from difference sources: CrHis(mix), CrHis purified isomer 1 (≧80% (w/w) purified; “CrHis-1”), CrHis purified isomer 2, or CrHis purified isomer 3, prepared as described herein. Insulin levels in the CrHis-1 group were significantly lower than other treatment groups (p=0.047) as shown in FIG. 8. In a similar study testing CrHis(mix) vs. semi-purified CrHis-1 (36% (w/w)), CrHis-2 (25% (w/w)), there were no differences in insulin levels between groups.

### Example 5

### Glucose Transporter (GLUT) Levels in Ovarian Tissue

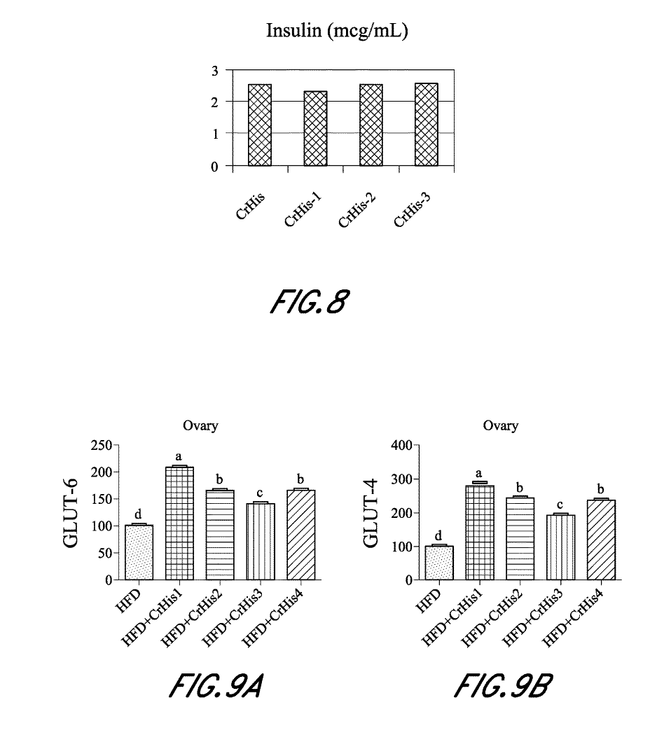

Female rats were fed a high-fat diet to induce insulin resistance. The rats were fed the same amount of Cr, but from difference sources: CrHis(mix) or CrHis purified isomer 1 (≧80% (w/w); “CrHis-1”). Expression of GLUT-6 and GLUT-4 was significantly higher in the CrHis-1 group as shown in FIGS. 9A-9B. In a similar study testing CrHis(mix) vs. semi-purified CrHis-1 (36% (w/w)), CrHis-2 (25% (w/w)), there were no differences in GLUT levels between groups.

### Example 6

### Tissue and Serum Chromium Levels

Rats were fed a high-fat diet to induce insulin resistance. Rats were fed the same amount of Cr, but from difference sources: CrHis(mix), CrHis(mid) (25-36% pure (w/w)), or CrHis(pure) (≧80% (w/w) pure of CrHis isomer 1; “CrHis-1). Tissue chromium levels for CrHis-1 (pure) were consistently greater than CrHis(mix), as shown in the Table 10 below.

TABLE 10

| | | Cr - | Cr - | Cr - | Cr - | Cr - |
|---|---|---|---|---|---|---|
| Study | Group | Serum | Liver | Kidney | Brain | Ovary |
| 1 | CrHis-1 (pure) | 0.090 | 1.230 | 1.256 | 0.464 | 0.272 |
| 1 | CrHis-2 (pure) | 0.084 | 1.156 | 1.205 | 0.434 | 0.256 |
| 1 | CrHis-3 (pure) | 0.081 | 1.092 | 1.079 | 0.425 | 0.250 |
| 1 | CrHis | 0.087 | 1.148 | 1.184 | 0.454 | 0.263 |
| 1 | Control (HFD) | 0.054 | 0.651 | 0.616 | 0.245 | 0.151 |
| 2 | CrHis-1 (mid) | 0.071 | 0.844 | 0.836 | 0.413 | 0.255 |
| 2 | CrHis-2 (mid) | 0.070 | 0.829 | 0.861 | 0.369 | 0.249 |
| 2 | CrHis | 0.080 | 0.869 | 0.843 | 0.425 | 0.258 |
| 2 | Control (HFD) | 0.046 | 0.623 | 0.574 | 0.215 | 0.092 |

### Example 7

### Evaluation of Isolated CrHis Isomers

Individuals (n=30) are divided into three groups (n=10), a placebo group, a group receiving a previously known composition of CrHis, and a group receiving an equivalent amount of chromium in the form of an isolated CrHis isomer. Surprisingly, one of the isolated CrHis isomers performs unexpectedly better than the other isomers, and better than previous CrHis compositions in treating the conditions and disorders described herein. This better performance is evidenced by achieving a greater therapeutic effect from the same amount of chromium, by providing a longer-lasting therapeutic effect, by achieving sustained therapeutic blood levels of chromium, and/or by achieving an equivalent therapeutic effect with either a smaller dose of chromium, or a reduced number of treatments.

Particular forms of CrHis (or combinations thereof) are found to have greater activity than prior formulations of chromium and histidinate. Greater activity may include an improved effect on cells and/or tissues. For example, certain forms of CrHis complexes may cause cells to uptake glucose faster and/or to a greater extent than other forms, may cause Cr concentrations in the blood, cells, and/or tissues to rise faster and/or to a greater extent than other forms, and/or may cause glucose transporters to increase activity and/or increase in concentration faster and/or to a greater extent than other forms.

Particular formulations having increased percentages of one or more isomers of CrHis (or combinations thereof) are found to have greater activity than prior formulations of chromium and histidinate. For example, a form having at least 40% (w/w) isomer 3 is found to have greater activity than forms that contained less than 30% (w/w) of isomer 3. In other examples, particular formulations having less than 20% (w/w) free histidine are found to have greater activity than forms that contained more than 20% (w/w) free histidine.

Greater activity may include an improved effect on cells and/or tissues. For example, certain formulations of CrHis complexes may cause cells to uptake glucose faster and/or to a greater extent than other forms, may cause Cr concentrations in the blood, cells, and/or tissues to rise faster and/or to a greater extent than other forms, and/or may cause glucose transporters to increase activity and/or increase in concentration faster and/or to a greater extent than other forms.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention. Moreover, the discussion of information in the Background section of the present application is not an admission that any of this information is prior art.

What is claimed is:

1. A formulation comprising:
at least about 5% (w/w) chromium histidinate isomer 1, having the structure: 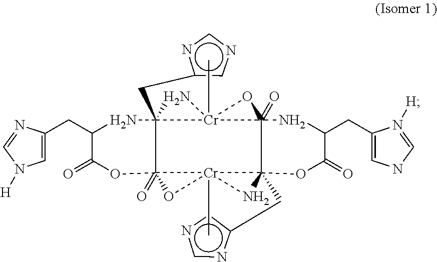
at least about 15% (w/w) chromium histidinate isomer 2, having the structure: 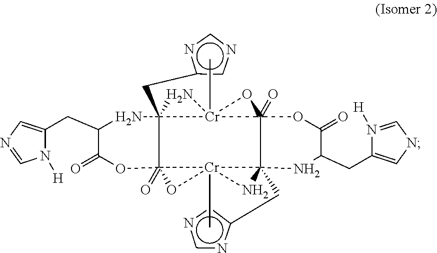
and
at least about 45% (w/w) chromium histidinate isomer 3, having the structure:

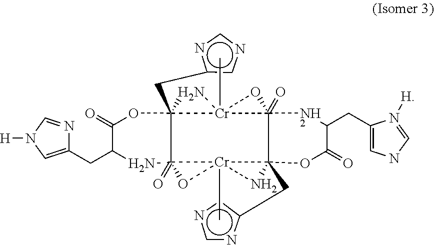

2. The formulation of claim 1, wherein the formulation contains no more than about 30% (w/w) free histidine.

3. The formulation of claim 1, wherein the formulation contains no more than about 20% (w/w) free histidine.

4. The formulation of claim 1, wherein the formulation contains no more than about 10% (w/w) free histidine.

5. The formulation of claim 1, wherein the formulation contains no more than about 8% (w/w) free histidine.

6. The formulation of claim 1, wherein the formulation comprises about 8% of chromium histidinate isomer 1, about 20% of chromium histidinate isomer 2, about 50% of chromium histidinate isomer 3, and not more than about 8% free histidine.

7. The formulation of claim 1, further comprising chromium picolinate.

8. The formulation of claim 1, further comprising at least one nutritionally acceptable carrier, excipient, or diluent.

9. A formulation comprising:
no more than 10% (w/w) chromium histidinate isomer 1, having the structure:

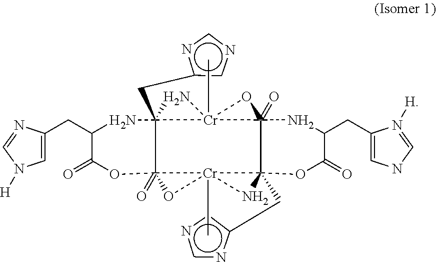

10. The formulation of claim 9, wherein the formulation contains no more than 10% (w/w) free histidine.

11. The formulation of claim 9, wherein the formulation contains no more than 8% (w/w) free histidine.

12. The formulation of claim 9, wherein the formulation further comprises at least 15% (w/w) chromium histidinate isomer 2, having the structure:

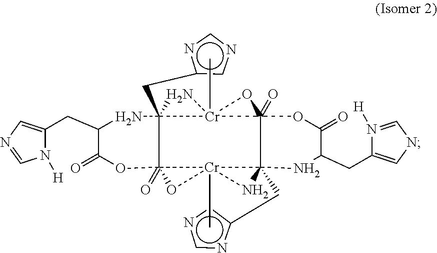

13. The formulation of claim 12, wherein the formulation further comprises at least 30% (w/w) chromium histidinate isomer 3, having the structure:

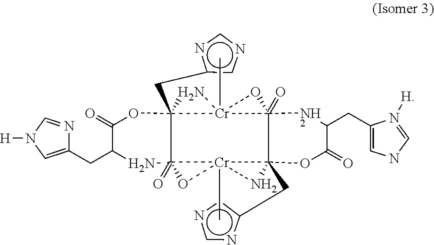

14. The formulation of claim 9, further comprising at least one nutritionally acceptable carrier, excipient, or diluent.

15. The formulation of claim 13, further comprising at least one nutritionally acceptable carrier, excipient, or diluent.

16. The formulation of claim 9, wherein the formulation includes at least 40% (w/w) chromium histidinate isomer 3.
